SEQUENCE LISTING (12 sequences)

SEQ ID NO: 1 - DNA, 1448 bp, Homo sapiens
ggtaccagaattcctcaccatgggctggtcctgcatcatcctgtttctggtggccacagccacaggggtgcacagccaggtgcagctggtgcagtctggggctgaggtgaagaagccaggggcctctgtgaaggtgtcctgcaagggctctggctacaccttcaccagctactggatgcactgggtgcgccaggccccaggccagaggctggagtggatcggggagatcgacccctctgagagcaacaccaactacaaccagaagttcaagggcagggtgaccctgacagtggacatctctgccagcacagcctacatggagctgtccagcctgagatctgaggacacagctgtgtactactgtgccagggggggatatgatggctgggactatgccatcgactactggggccagggcaccctggtgacagtcagctcagccagcaccaagggcccctctgtgttccccctggcccccagcagcaagagcacctctgggggcacagctgccctgggctgcctggtgaaggactacttcccagagccagtgacagtgtcctggaactctggggccctgacatctggggtgcacaccttccctgctgtgctgcagtcctctggcctgtatagcctgtcctctgtggtgacagtgcccagcagcagcctgggcacccagacctacatctgcaatgtgaaccacaagcccagcaacacaaaggtggacaagaaggtggagcccaagagctgtgacaagacccacacctgccccccctgcccagccccagagctggctggggctccatctgtgtttctgttcccccccaagcccaaggacaccctgatgatcagcaggaccccagaggtgacctgtgtggtggtggatgtgagccatgaggatccagaggtgaagttcaattggtatgtggatggggtggaggtgcacaatgccaagaccaagcccagagaggagcagtacaacagcacctacagggtggtgtctgtgctgacagtgctgcaccaggactggctgaatggcaaggagtacaagtgcaaggtctccaataaggccctgccagcccccatcgaaaagaccatcagcaaggccaagggccagccaagggagccccaggtgtacaccctgcccccctccagagatgagctgaccaagaaccaggtgtccctgacctgtctggtgaagggcttctacccctctgacatcgctgtggagtgggagagcaatggccagccagagaacaactacaagaccacccccccagtgctggattctgatggcagcttcttcctgtacagcaagctgacagtggataagtccaggtggcagcagggcaatgtgttcagctgctctgtgatgcatgaggccctgcacaaccactacacccagaagagcctgtccctgagcccaggcaagtgatagtctagaggagctc

SEQ ID NO: 2 - DNA, 754 bp, Homo sapiens
ggtaccagcggccgcctcaccatgggctggtcctgcatcatcctgtttctggtggccacagccacaggggtgcactctgatgtggtgatgacccagagccccctgagcctgccagtgaccccaggggagccagccagcatcagctgcaggtccagccagagcctggccaagagctatggcaacacctacctgagctggtatctgcagaagccaggccagtccccccagctgctgatctatggcatcagcaacaggttctctggggtgccagacagattctctggctctgggtctggcacagacttcaccctgaagatcagcagggtggaggctgaggatgtgggggtgtactactgcctgcagggcacacaccagccctacacctttggccagggcaccaaggtggagatcaagcgtacggtggctgccccctctgtgttcatcttccccccctctgatgagcagctgaagtctgggacagcctctgtggtgtgcctgctgaacaacttctaccccagggaggccaaggtgcagtggaaggtggacaatgccctgcagtctggcaacagccaggagtctgtgacagagcaggacagcaaggactccacctacagcctgagcagcaccctgaccctgagcaaggctgactatgagaagcacaaggtgtacgcctgtgaggtgacccaccagggcctgtccagcccagtgaccaagagcttcaacaggggggagtgctgatagtctagaggagctc

SEQ ID NO: 3 - DNA, 1447 bp, Homo sapiens
ggtaccgaattcctcaccatgggctggtcctgcatcatcctgtttctggtggccaccgccaccggcgtgcacagccaggtgcagctggtgcagagcggagccgaggtgaagaagccaggcgccagcgtcaaggtgtcctgcaagggcagcggctacaccttcaccagctactggatgcactgggtgcggcaggccccaggccagaggctggagtggatcggcgagatcgaccccagcgagagcaacaccaactacaaccagaagttcaagggcagggtcaccctgaccgtggacatcagcgccagcaccgcctacatggagctgtccagcctgagaagcgaggacaccgccgtgtactactgcgccaggggcggctacgacggctgggactacgccatcgactactggggccagggcaccctggtgaccgtcagctcagccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggggtgcacaccttccccgccgtgctgcagagcagcggcctgtacagcctgtccagcgtggtgacagtgcccagcagcagcctgggcacccagacctacatctgcaacgtgaaccacaagccctccaacaccaaggtggacaagaaggtggagcccaagagctgcgacaagacccacacctgccccccctgcccagcccccgagctggccggagccccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgcctgcccccatcgaaaagaccatcagcaaggccaagggccagccacgggagccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaagtgatagtctagaggagctc

SEQ ID NO: 4 - DNA, 753 bp, Homo sapiens
ggtaccgcggccgcctcaccatgggctggtcctgcatcatcctgtttctggtggccaccgccaccggcgtgcacagcgacgtggtgatgacccagagccccctgagcctgcccgtgaccccaggcgagcccgccagcatcagctgcaggtccagccagagcctggccaagagctacggcaacacctacctgagctggtatctgcagaagcccggccagtccccccagctgctgatctacggcatcagcaacaggttcagcggcgtgcccgacaggttctccggcagcggctccggcaccgacttcaccctgaaaatcagcagggtggaggccgaggacgtgggcgtgtactactgcctgcagggcacacaccagccctacaccttcggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctgatagtctagaggagctc

SEQ ID NO: 5 - DNA, 1448 bp, Homo sapiens
gagctcctctagactatcacttgcctgggctcagggacaggctcttctgggtgtagtggttgtgcagggcctcatgcatcacagagcagctgaacacattgccctgctgccacctggacttatccactgtcagcttgctgtacaggaagaagctgccatcagaatccagcactgggggggtggtcttgtagttgttctctggctggccattgctctcccactccacagcgatgtcagaggggtagaagcccttcaccagacaggtcagggacacctggttcttggtcagctcatctctggaggggggcagggtgtacacctggggctcccttggctggcccttggccttgctgatggtcttttcgatgggggctggcagggccttattggagaccttgcacttgtactccttgccattcagccagtcctggtgcagcactgtcagcacagacaccaccctgtaggtgctgttgtactgctcctctctgggcttggtcttggcattgtgcacctccaccccatccacataccaattgaacttcacctctggatcctcatggctcacatccaccaccacacaggtcacctctggggtcctgctgatcatcagggtgtccttgggcttgggggggaacagaaacacagatggagccccagccagctctggggctgggcagggggggcaggtgtgggtcttgtcacagctcttgggctccaccttcttgtccacctttgtgttgctgggcttgtggttcacattgcagatgtaggtctgggtgcccaggctgctgctgggcactgtcaccacagaggacaggctatacaggccagaggactgcagcacagcagggaaggtgtgcaccccagatgtcagggccccagagttccaggacactgtcactggctctgggaagtagtccttcaccaggcagcccagggcagctgtgcccccagaggtgctcttgctgctgggggccagggggaacacagaggggcccttggtgctggctgagctgactgtcaccagggtgccctggccccagtagtcgatggcatagtcccagccatcatatccccccctggcacagtagtacacagctgtgtcctcagatctcaggctggacagctccatgtaggctgtgctggcagagatgtccactgtcagggtcaccctgcccttgaacttctggttgtagttggtgttgctctcagaggggtcgatctccccgatccactccagcctctggcctggggcctggcgcacccagtgcatccagtagctggtgaaggtgtagccagagcccttgcaggacaccttcacagaggcccctggcttcttcacctcagccccagactgcaccagctgcacctggctgtgcacccctgtggctgtggccaccagaaacaggatgatgcaggaccagcccatggtgaggaattctggtacc

SEQ ID NO: 6 - DNA, 754 bp, Homo sapiens
gagctcctctagactatcagcactcccccctgttgaagctcttggtcactgggctggacaggccctggtgggtcacctcacaggcgtacaccttgtgcttctcatagtcagccttgctcagggtcagggtgctgctcaggctgtaggtggagtccttgctgtcctgctctgtcacagactcctggctgttgccagactgcagggcattgtccaccttccactgcaccttggcctccctggggtagaagttgttcagcaggcacaccacagaggctgtcccagacttcagctgctcatcagagggggggaagatgaacacagagggggcagccaccgtacgcttgatctccaccttggtgccctggccaaaggtgtagggctggtgtgtgccctgcaggcagtagtacacccccacatcctcagcctccaccctgctgatcttcagggtgaagtctgtgccagacccagagccagagaatctgtctggcaccccagagaacctgttgctgatgccatagatcagcagctggggggactggcctggcttctgcagataccagctcaggtaggtgttgccatagctcttggccaggctctggctggacctgcagctgatgctggctggctcccctggggtcactggcaggctcagggggctctgggtcatcaccacatcagagtgcacccctgtggctgtggccaccagaaacaggatgatgcaggaccagcccatggtgaggcggccgctggtacc

SEQ ID NO: 7 - DNA, 1447 bp, Homo sapiens
gagctcctctagactatcacttgccgggggacaggctcaggctcttctgggtgtagtggttgtgcagggcctcgtgcatcacgctgcagctgaacacgttgccctgctgccacctgctcttgtccactgtcagcttgctgtacaggaagaagctgccgtcgctgtccagcactgggggggtggtcttgtagttgttctcgggctggccgttgctctcccactccacggcgatgtcgctggggtagaagcccttcaccagacaggtcagggacacctggttcttggtcagctcgtcccgggaggggggcagggtgtacacctggggctcccgtggctggcccttggccttgctgatggtcttttcgatgggggcaggcagggccttgttggagaccttgcacttgtattccttgccgttcagccagtcctggtgcagcacggtcagcacggacaccaccctgtaggtgctgttgtactgctcctctctgggcttggtcttggcgttgtgcacctccacgccgtccacgtaccagttgaacttcacctctgggtcctcgtggctcacgtccaccaccacgcaggtcacctcgggggtcctgctgatcatcagggtgtccttgggcttgggggggaacaggaacacggagggggctccggccagctcgggggctgggcagggggggcaggtgtgggtcttgtcgcagctcttgggctccaccttcttgtccaccttggtgttggagggcttgtggttcacgttgcagatgtaggtctgggtgcccaggctgctgctgggcactgtcaccacgctggacaggctgtacaggccgctgctctgcagcacggcggggaaggtgtgcaccccgctggtcagggctccgctgttccaggacacggtcacgggctcggggaagtagtccttcaccaggcagcccagggcggctgtgccgccgctggtgctcttgctgctgggggccagggggaacacgctggggcccttggtgctggctgagctgacggtcaccagggtgccctggccccagtagtcgatggcgtagtcccagccgtcgtagccgcccctggcgcagtagtacacggcggtgtcctcgcttctcaggctggacagctccatgtaggcggtgctggcgctgatgtccacggtcagggtgaccctgcccttgaacttctggttgtagttggtgttgctctcgctggggtcgatctcgccgatccactccagcctctggcctggggcctgccgcacccagtgcatccagtagctggtgaaggtgtagccgctgcccttgcaggacaccttgacgctggcgcctggcttcttcacctcggctccgctctgcaccagctgcacctggctgtgcacgccggtggcggtggccaccagaaacaggatgatgcaggaccagcccatggtgaggaattcggtacc

SEQ ID NO: 8 - DNA, 753 bp, Homo sapiens
gagctcctctagactatcagcactcgcccctgttgaagctcttggtcacggggctggacaggccctggtgggtcacctcgcaggcgtacaccttgtgcttctcgtagtcggccttgctcagggtcagggtgctgctcaggctgtaggtggagtccttgctgtcctgctcggtgacgctctcctggctgttgccgctctgcagggcgttgtccaccttccactgcaccttggcctcccgggggtagaagttgttcagcaggcacaccacgctggcggtgccgctcttcagctgctcgtcgctgggggggaagatgaacacgctgggagcggccaccgtacgcttgatttccaccttggtgccctggccgaaggtgtagggctggtgtgtgccctgcaggcagtagtacacgcccacgtcctcggcctccaccctgctgattttcagggtgaagtcggtgccggagccgctgccggagaacctgtcgggcacgccgctgaacctgttgctgatgccgtagatcagcagctggggggactggccgggcttctgcagataccagctcaggtaggtgttgccgtagctcttggccaggctctggctggacctgcagctgatgctggcgggctcgcctggggtcacgggcaggctcagggggctctgggtcatcaccacgtcgctgtgcacgccggtggcggtggccaccagaaacaggatgatgcaggaccagcccatggtgaggcggccgcggtacc

SEQ ID NO: 9 - DNA, 733 bp, Homo sapiens
  variation 39: n = a or c
atgggctggtcctgcatcatcctgtttctggtggccacngccacnggngtgcacnnngangtggtgatgacccagagccccctgagcctgccngtgaccccaggngagccngccagcatcagctgcaggtccaggcagagcctggccaagagctanggcaacacctacctgagccggtatctgcagaagccnggccagtcgccccagctgctgatctanggcatcagcaacaggttcnnnggngtgccngacagnttctcnggcnnnggntcnggcacngacttcaccctgaanatcagcagggtggaggcngaggangtgggngtgtactactgcctgcagggcacacaccagccctacaccttnggccagggcaccaaggtgganatcaagcgtacggtggcngcncccnnngtgttcatcttcccccccnnngangagcagctgaagnnnggnacngccnnngtggtgtgcctgctgaacaacttctaccccngggaggccaaggtgcagtggaaggtggacaangccctgcagnnnggcaacagccaggagnnngtnacngagcaggacagcaaggactccacctacagcctgagcagcaccctgaccctgagcaaggcngactangagaagcacaaggtgtacgcctgngaggtgacccaccagggcctgtccagcccngtgaccaagagcttcaacaggggngagtgctgatagtctagaggagctc

SEQ ID NO: 10 - DNA, 1429 bp, Homo sapiens
  variation 39: n = a or c
atgggctggtcctgcatcatcctgtttctggtggccacngccacnggngtgcacagccaggtgcagctggtgcagnnnggngcngaggtgaagaagccaggngccnnngtnaaggtgtcctgcaagggcnnnggctacaccttcaccagctactggatgcactgggtgcgncaggccccaggccagaggctggagtggatcggngagatcgaccccnnngagagcaacaccaactacaaccagaagttcaagggcagggtnaccctgacngtggacatcnnngccagcacngcctacatggagctgtccagcctgagannngaggacacngcngtgtactactgngccaggggnggntanganggctgggactangccatcgactactggggccagggcaccctggtgacngtcagctcagccagcaccaagggccccnnngtgttccccctggcccccagcagcaagagcaccnnnggnggcacagcngccctgggctgcctggtgaaggactacttcccngagccngtgacngtgtcctggaacnnnggngccctgacnnnnggggtgcacaccttcccngcngtgctgcagnncnnnggcctgtanagcctgtccnnngtggtgacagtgcccagcagcagcctgggcacccagacctacatctgcaangtgaaccacaagcccnncaacacnaaggtggacaagaaggtggagcccaagagctgngacaagacccacacctgccccccctgcccagccccngagctggcnggngcnccntcngtgttnctgttcccccccaagcccaaggacaccctgatgatcagcaggaccccngaggtgacctgngtggtggtggangtgagccangagganccagaggtgaagttcaantggtangtgganggngtggaggtgcacaangccaagaccaagcccagagaggagcagtacaacagcacctacagggtggtgtcngtgctgacngtgctgcaccaggactggctgaanggcaaggantacaagtgcaaggtctccaanaaggccctgccngcccccatcgaaaagaccatcagcaaggccaagggccagccangggagccccaggtgtacaccctgcccccctccngngangagctgaccaagaaccaggtgtccctgacctgtctggtgaagggcttctaccccnnngacatcgcngtggagtgggagagcaanggccagccngagaacaactacaagaccacccccccagtgctggannnnganggcagcttcttcctgtacagcaagctgacagtgganaagnncaggtggcagcagggcaangtgttcagctgcnnngtgatgcangaggccctgcacaaccactacacccagaagagcctgnncctgnncccnggcaagtgatagtctagaggagctc

SEQ ID NO: 11 - protein, 238 aa, Homo sapiens
MGWSCIILFLVATATGVHSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 470 aa, Homo sapiens
MGWSCIILFLVATATGVHSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2010/0297699 A1
(43) Pub. Date: Nov. 25, 2010

(54) Nucleic Acids Encoding Humanized Immunoglobulin That Binds Alpha4Beta7 Integrin

(75) Inventors: Ping Li, Wellesley, MA (US); Marcus Graf, Regensburg (DE)

Correspondence Address: McDermott Will & Emery, 600 13th Street, NW, Washington, DC, 20005-3096, US

(73) Assignee: Millennium Pharmaceuticals, Inc., Cambridge, MA (US)

(21) Appl. No.: 12/531,534

(22) PCT Filed: Mar. 19, 2008
(86) PCT No.: PCT/US2008/003560

§ 371 Date: Sep. 16, 2009

Related U.S. Application Data
(60) Provisional application No. 60/918,944, filed on Mar. 20, 2007.

(51) Int. Cl. C12P 21/04; C07H 21/04; C12N 15/63; C12N 5/10

(52) U.S. Cl. 435/69.6; 536/23.53; 435/320.1; 435/328

(57) ABSTRACT

The invention relates to an isolated nucleic acid encoding a humanized immunoglobulin that has binding specificity for α4β7 integrin and comprises the complementarity determining regions (CDRs) of mouse Act-1 antibody. The present invention further relates to an isolated nucleic acid encoding a humanized heavy chain and an isolated nucleic acid encoding a humanized light chain. The invention also relates to recombinant vectors and host cells that comprise a nucleic acid which encodes a humanized immunoglobulin, humanized immunoglobulin heavy chain or a humanized immunoglobulin light chain, and to methods of preparing a humanized immunoglobulin.

### RELATED APPLICATION

This application claims the benefit of U.S. Provisional Application No. 60/918,944, filed on Mar. 20, 2007. The entire teachings of the above application are incorporated herein by reference.

### BACKGROUND OF THE INVENTION

Integrin receptors are important for regulating both lymphocyte recirculation and recruitment to sites of inflammation (Carlos, T. M. and Harlan, J. M., Blood, 84:2068-2101 (1994)). The human α4β7 integrin has several ligands, one of which is the mucosal vascular addressin MAdCAM-1 (Berlin, C., et al., Cell 74: 185-195 (1993); Erie, D. J., et al., J. Immunol. 153517-528 (1994)), which is expressed on high endothelial venules in mesenteric lymph nodes and Peyer's patches (Streeter, P. R., et al., Nature 331:41-46 (1998)). The α4β7 integrin acts as a homing receptor that mediates lymphocyte migration to intestinal mucosal lymphoid tissue (Schweighoffer, T., et al. J. Immunol. 151:717-729 (1993)). In addition, the α4β7 integrin interacts with fibronectin and vascular cell adhesion molecule-1 (VCAM-1).

Inflammatory bowel disease (IBD), such as ulcerative colitis and Crohn's disease, for example, can be a debilitating and progressive disease involving inflammation of the gastrointestinal tract. IBD treatments have included anti-inflammatory drugs (such as, corticosteroids and sulfasalazine), immunosuppressive drugs (such as, 6-mercaptopurine, cyclosporine and azathioprine) and surgery (such as, colectomy). Podolsky, New Engl. J. Med., 325928-937 (1991) and Podolsky, New Engl. J. Med., 325:1008-1016 (1991).

Antibodies against human α4β7 integrin, such as murine monoclonal antibody Act-1 (mAb Act-1), interfere with α4β7 integrin binding to mucosal addressin cell adhesion molecule-1 (MAdCAM-1) present on high endothelial venules in mucosal lymph nodes. Act-1 was originally isolated by Lazarovits, A. I., et al., J. Immunol. 133:1857-1862 (1984). Humanized Act-1 antibodies have been et al., J. Immunol. 133:1857-1862 (1984). Humanized Act-1 antibodies have been prepared which can be administered to humans to treat diseases, such as inflammatory bowel disease. (See, e.g., U.S. Pat. No. 7,147,851 and U.S. application Ser. No. 11/599,151). Humanized antibodies are generally produced by expression of recombinant constructs that encode the heavy and light chains in a mammalian host cell. This method of production has the benefit of yielding antibodies that are correctly assembled and folded. However, expression yields in mammalian systems are frequently low and large cultures must be processed to recover sufficient quantities of antibody, thereby increasing the cost of antibody production. Thus, a need exists for improved constructs and methods for making humanized antibodies.

### SUMMARY OF THE INVENTION

The invention relates to isolated nucleic acids that encode the humanized antibody MLN02, the humanized heavy chain of the humanized antibody MLN02 and/or the humanized light chain of the humanized antibody MLN02. In some embodiments, the invention is an isolated nucleic acid encoding the humanized antibody MLN02. The isolated nucleic acid can comprise a first nucleotide sequence that encodes the humanized light chain and comprises nucleotides 58-714 of SEQ ID NO:9, with the proviso that the first nucleotide sequence encodes amino acids 20-238 of SEQ ID NO:11; and a second nucleotide sequence that encodes the humanized heavy chain and comprises nucleotides 58-1410 of SEQ ID NO:10, with the proviso that the second nucleotide sequence encodes amino acids 20-470 of SEQ ID NO:12. In some embodiments, the first nucleotide sequence comprises nucleotides 77-1429 of SEQ ID NO:1 or nucleotides 76-1428 of SEQ ID NO:3, and/or the second nucleotide sequence comprises nucleotides 79-735 of SEQ ID NO:2 or nucleotides 78-734 of SEQ ID NO:4.

The invention also relates to an isolated nucleic acid encoding the humanized light chain of the humanized antibody MLN02. The isolated nucleic acid can comprise nucleotides 58-714 of SEQ ID NO:9, with the proviso that the nucleotides encode amino acids 20-238 of SEQ ID NO:11. In one embodiment, the isolated nucleic acid comprises nucleotides 79-735 of SEQ ID NO:2 or nucleotides 78-734 of SEQ ID NO:4. Preferably, the isolated nucleic acid comprises nucleotides 79-735 of SEQ ID NO:2.

The invention also relates to an isolated nucleic acid encoding the humanized immunoglobulin heavy chain of the humanized antibody MLN02. The isolated nucleic acid can comprise nucleotides 58-1410 of SEQ ID NO:10, with the proviso that the nucleotides encode amino acids 20-470 of SEQ ID NO:12. In one embodiment, the isolated nucleic acid comprises nucleotides 77-1429 of SEQ ID NO:1 or nucleotides 76-1428 of SEQ ID NO:3. Preferably, the isolated nucleic acid comprises nucleotides 77-1429 of SEQ ID NO:1.

The invention also relates to recombinant vectors (e.g., expression vectors, mammalian cell expression vectors) that comprise a nucleic acid encoding the humanized antibody MLN02 (humanized light chain and humanized heavy chain), the humanized heavy chain of the humanized antibody MLN02, or the humanized light chain of the humanized antibody MLN02. In some embodiments, the recombinant vector comprises a first nucleotide sequence that encodes the humanized light chain and comprises nucleotides 58-714 of SEQ ID NO:9, with the proviso that the first nucleotide sequence encodes amino acids 20-238 of SEQ ID NO:11; and a second nucleotide sequence that encodes the humanized heavy chain and comprises nucleotides 58-1410 of SEQ ID NO:10, with the proviso that the second nucleotide sequence encodes amino acids 20-470 of SEQ ID NO:12. In other embodiments, the recombinant vector comprises an isolated nucleic acid that encodes the humanized immunoglobulin light chain of the humanized antibody MLN02 and comprises nucleotides 58-714 of SEQ ID NO:9, with the proviso that the nucleotides encode amino acids 20-238 of SEQ ID NO:11. In further embodiments, the recombinant vector comprises an isolated nucleic acid that encodes the humanized immunoglobulin heavy chain of the humanized antibody MLN02 comprises nucleotides 58-1410 of SEQ ID NO:10, with the proviso that the nucleotides encode amino acids 20-470 of SEQ ID NO:12.

In some embodiments, the invention is a recombinant vector comprising an isolated nucleic acid encoding the humanized immunoglobulin heavy chain of the humanized antibody MLN02 and comprises nucleotides 77-1429 of SEQ ID NO:1 or nucleotides 76-1428 of SEQ ID NO:3.

In some embodiments, the invention is a recombinant vector comprising an isolated nucleic acid encoding the humanized immunoglobulin light chain of the humanized antibody MLN02 and comprises nucleotides 79-735 of SEQ ID NO:2 or nucleotides 78-734 of SEQ ID NO:4.

In some embodiments, the invention is a recombinant vector encoding the humanized antibody MLN02, wherein the recombinant vector comprises a first nucleic acid that encodes the humanized immunoglobulin heavy chain and a second nucleic acid that encodes the humanized immunoglobulin light chain, wherein the first nucleic acid comprises nucleotides 77-1429 of SEQ ID NO:1, and the second nucleic acid comprises nucleotides 79-735 of SEQ ID NO:2.

In some embodiments, the invention is a recombinant vector encoding the humanized antibody MLN02, wherein the recombinant vector comprises a first nucleic acid that encodes the humanized immunoglobulin heavy chain and a second nucleic acid that encodes the humanized immunoglobulin light chain, wherein the first nucleic acid comprises nucleotides 76-1428 of SEQ ID NO:3, and the second nucleic acid comprises nucleotides 78-734 of SEQ ID NO:4.

The invention also relates to an isolated host cell that comprises an isolated nucleic acid that encodes the humanized antibody MLN02 (humanized light chain and humanized heavy chain), the humanized heavy chain of the humanized antibody MLN02, or the humanized light chain of the humanized antibody MLN02. For example, in some embodiments, the isolated host cell comprises a recombinant vector (e.g., expression vector, mammalian expression vector) of the invention.

In some embodiments, the isolated host cell comprises an isolated nucleic acid encoding the humanized antibody MLN02, the nucleic acid comprises a first nucleotide sequence that comprises nucleotides 58-714 of SEQ ID NO:9, with the proviso that the first nucleotide sequence encodes amino acids 20-238 of SEQ ID NO:11; and a second nucleotide sequence that comprises nucleotides 58-1410 of SEQ ID NO:10, with the proviso that the second nucleotide sequence encodes amino acids 20-470 of SEQ ID NO:12.

In some embodiments, the isolated host cell comprises a recombinant vector encoding the humanized antibody MLN02. The recombinant vector comprises a first nucleotide sequence that comprises nucleotides 58-714 of SEQ ID NO:9, with the proviso that the first nucleotide sequence encodes amino acids 20-238 of SEQ ID NO:11; and a second nucleotide sequence that comprises nucleotides 58-1410 of SEQ ID NO:10, with the proviso that the second nucleotide sequence encodes amino acids 20-470 of SEQ ID NO:12.

In some embodiments, the isolated host cell comprises an isolated nucleic acid encoding the humanized light chain of the humanized antibody MLN02, wherein the isolated nucleic acid comprises nucleotides 58-714 of SEQ ID NO:9, with the proviso that the nucleotides encode amino acids 20-238 of SEQ ID NO:11.

In some embodiments, the isolated host cell comprises a recombinant vector comprising an isolated nucleic acid encoding the humanized light chain of the humanized antibody MLN02, wherein the isolated nucleic acid comprises nucleotides 58-714 of SEQ ID NO:9, with the proviso that the nucleotides encode amino acids 20-238 of SEQ ID NO:11.

In some embodiments, the isolated host cell comprises an isolated nucleic acid encoding the humanized immunoglobulin heavy chain of the humanized antibody MLN02, wherein the isolated nucleic acid comprises nucleotides 58-1410 of SEQ ID NO:10, with the proviso that the nucleotides encode amino acids 20-470 of SEQ ID NO:12.

In some embodiments, the isolated host cell comprises a recombinant vector comprising an isolated nucleic acid encoding the humanized immunoglobulin heavy chain of the humanized antibody MLN02, wherein the isolated nucleic acid comprises nucleotides 58-1410 of SEQ ID NO:10, with the proviso that the nucleotides encode amino acids 20-470 of SEQ ID NO:12.

The invention also relates to a method of producing the humanized antibody MLN02, comprising maintaining a host cell of the invention (e.g., a host cell that contains one or more isolated nucleic acids that encode humanized antibody MLN02 under conditions suitable for expression of the humanized antibody MLN02, whereby the chains of humanized antibody MLN02 are expressed and the humanized MLN02 is produced.

The invention also relates to a method of producing the humanized antibody MLN02 comprising providing and expressing an isolated nucleic acid of the invention (e.g., an isolated nucleic acid that encodes the humanized antibody MLN02 (e.g., the humanized light chain and the humanized heavy chain of MLN02), whereby the chains of humanized antibody MLN02 are expressed and the humanized antibody MLN02 is produced.

The invention also relates to a method of producing the humanized antibody MLN02 comprising providing a recombinant vector of the invention and expressing the recombinant vector, whereby the chains of humanized antibody MLN02 are expressed and the humanized antibody MLN02 is produced.

The invention also relates to methods for producing the light or heavy chain of MLN02. For example, the light or heavy chain of MLN02 can be produced by expression of an isolated nucleic acid (e.g., by maintaining a host cell under suitable conditions) as described herein.

### BRIEF DESCRIPTION OF THE DRAWINGS

FIG. 1A-1D is an illustration of the amino acid sequence of the heavy chain of a humanized Act-1 immunoglobulin referred to herein as MLN02 and of a nucleotide sequence encoding the heavy chain. The coding strand is SEQ ID NO:1 and the non-coding strand is SEQ ID NO:5. The open reading frame is nucleotides 20-1429 of SEQ ID NO:1. The nucleotide sequence contains an untranslated region (nucleotides 1-19 of SEQ ID NO:1) and encodes a signal peptide (nucleotides 20-76 of SEQ ID NO:1). The mature humanized heavy chain is encoded by nucleotides 77-1429 of SEQ ID NO:1. The deduced amino acid sequence of the heavy chain is SEQ ID NO:12. The signal peptide is amino acids 1-19 of SEQ ID NO:12 and the mature heavy chain is amino acids 20-470 of SEQ ID NO:12.

FIG. 2A-2B is an illustration of the amino acid sequence of the light chain of a humanized Act-1 immunoglobulin referred to herein as MLN02 and of a nucleotide sequence encoding the light chain. The coding strand is SEQ ID NO:2 and the non-coding strand is SEQ ID NO:6. The open reading frame is nucleotides 22-735 of SEQ ID NO:2. The nucleotide sequence contains an untranslated region (nucleotides 1-21 of SEQ ID NO:2) and encodes a signal peptide (nucleotides 22-78 of SEQ ID NO:2). The mature humanized light chain is encoded by nucleotides 79-735 of SEQ ID NO:2. The deduced amino acid sequence of the light chain is SEQ ID NO:11. The signal peptide is amino acids 1-19 of SEQ ID NO:11 and the mature light chain is amino acids 20-238 of SEQ ID NO:11.

FIG. 3A-3D is an illustration of the amino acid sequence of the heavy chain of a humanized Act-1 immunoglobulin referred to herein as MLN02 and of a nucleotide sequence encoding the heavy chain. The coding strand is SEQ ID NO:3 and the non-coding strand is SEQ ID NO:7. The open reading frame is nucleotides 19-1428 of SEQ ID NO:3. The nucleotide sequence encodes a signal peptide (nucleotides 19-75 of SEQ ID NO:3). The mature humanized heavy chain is encoded by nucleotides 76-1428 of SEQ ID NO:3. The deduced amino acid sequence of the heavy chain (SEQ ID NO:12) is also shown.

FIG. 4A-4B is an illustration of the amino acid sequence of the light chain of a humanized Act-1 immunoglobulin referred to herein as MLN02 and of a nucleotide sequence encoding the light chain. The coding strand is SEQ ID NO:4 and the non-coding strand is SEQ ID NO:8. The open reading frame is nucleotides 21-734 of SEQ ID NO:4. The nucleotide sequence encodes a signal peptide (nucleotides 21-77 of SEQ ID NO:4). The mature humanized light chain is encoded by nucleotides 78-734 of SEQ ID NO:4. The deduced amino acid sequence of the light chain (SEQ ID NO:11) is also shown.

FIG. 5A-5B is an alignment of nucleotide sequences that encode the humanized light chain of MLN02. The top sequence is the open reading frame of SEQ ID NO:2 (nucleotides 22-735 of SEQ ID NO:2), the middle sequence is the open reading frame of SEQ ID NO:4 (nucleotides 21-734 of SEQ ID NO:4) and the bottom sequence is a consensus sequence (SEQ ID NO:9). In SEQ ID NO:9, nucleotides 55, 238, 265, 412, 433, 451, 463, 538 and 556 are either A or T; nucleotides 48, 56, 105, 239, 243, 266, 270, 324, 413, 434, 452, 456, 464, 539, 557, 561 and 708 are either G or C; nucleotides 57, 60, 156, 219, 240, 267, 273, 312, 318, 366, 405, 414, 435, 438, 453, 465, 528, 540, 558, 624, 630 and 654 are either T or C; nucleotides 294 and 387 are either G or A; nucleotides 39, 45, 93, 111, 192, 279, 459, 496, 564 and 684 are either A or C

FIG. 6A-6C is an alignment of nucleotide sequences that encode the humanized heavy chain of MLN02. The top sequence is the open reading frame of SEQ ID NO:1 (nucleotides 20-1429 of SEQ ID NO:1), the middle sequence is the open reading frame of SEQ ID NO:3 (nucleotides 19-1428 of SEQ ID NO:3) and the bottom sequence is a consensus sequence (SEQ ID NO:10). In SEQ ID NO:10, nucleotides 39, 45, 270, 291, 330, 357, 411, 522, 528, 534, 561, 696, 765, 783, 840, 990, 1099, 1132, 1230, and 1404 are A or C; position 48, 77, 102, 107, 111, 131, 171, 204, 218, 261, 281, 320, 354, 440, 476, 480, 548, 563, 596, 599, 620, 689, 912, 1193, 1268, 1313, 1346, 1394, and 1400 are G or C; nucleotides 76, 106, 130, 217, 280, 319, 439, 475, 547, 562, 595, 598, 619, 688, 1056, 1192, 1267, 1312, 1345, and 1399 are T or A; nucleotides 78, 84, 108, 132, 219, 282, 321, 333, 345, 360, 363, 375, 441, 477, 489, 549, 564, 582, 585, 600, 609, 621, 672, 729, 774, 780, 786, 792, 852, 864, 873, 879, 897, 903, 909, 927, 981, 1014, 1044, 1137, 1194, 1203, 1221, 1266, 1269, 1272, 1308, 1332, 1347, and 1356 are T or C; and nucleotides 81, 552, 777, 1023, and 1134 are G or A.

FIG. 7 is schematic illustration of the Fluid Microvolume Assay Technology (FMAT) human Ig G1 Fc immunocompetition assay used to assess production of MLN02.

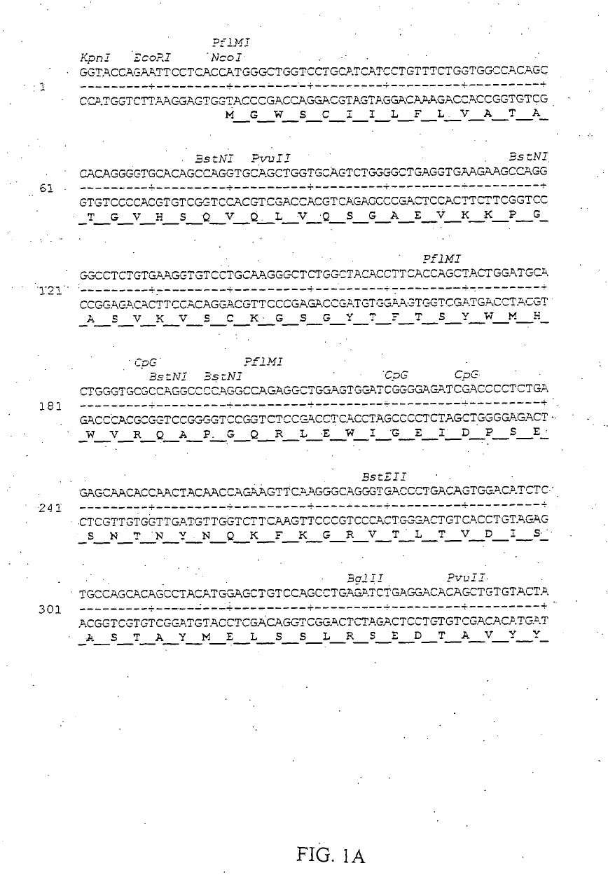

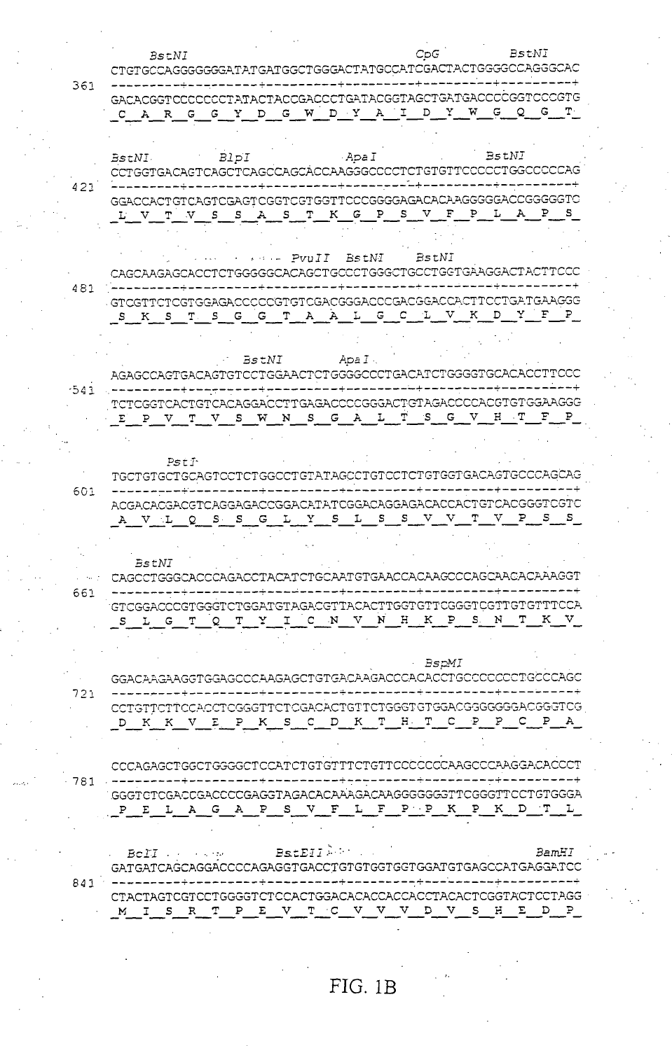

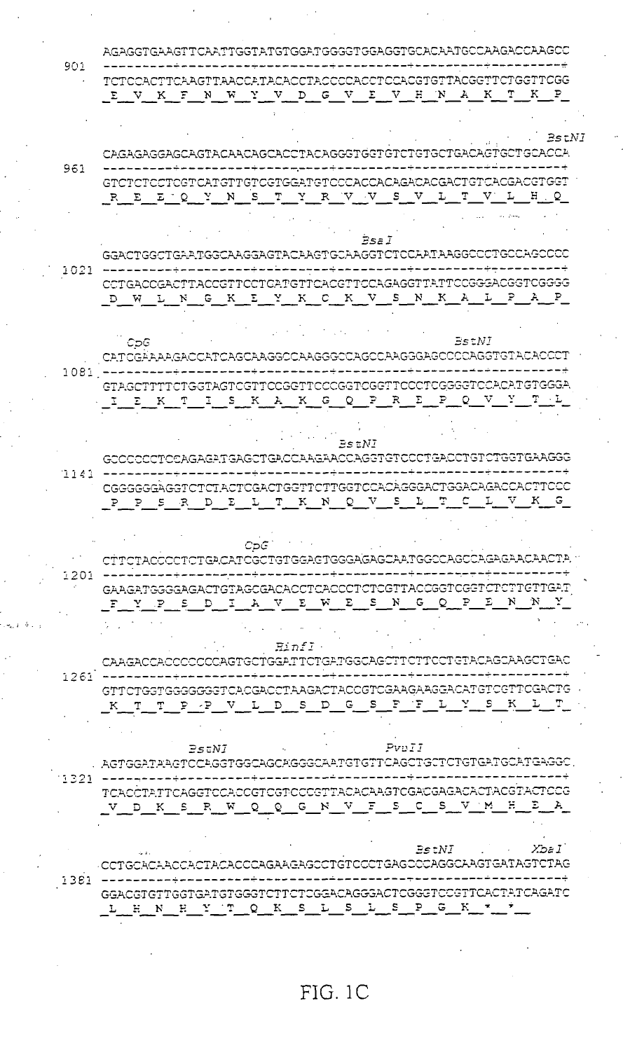

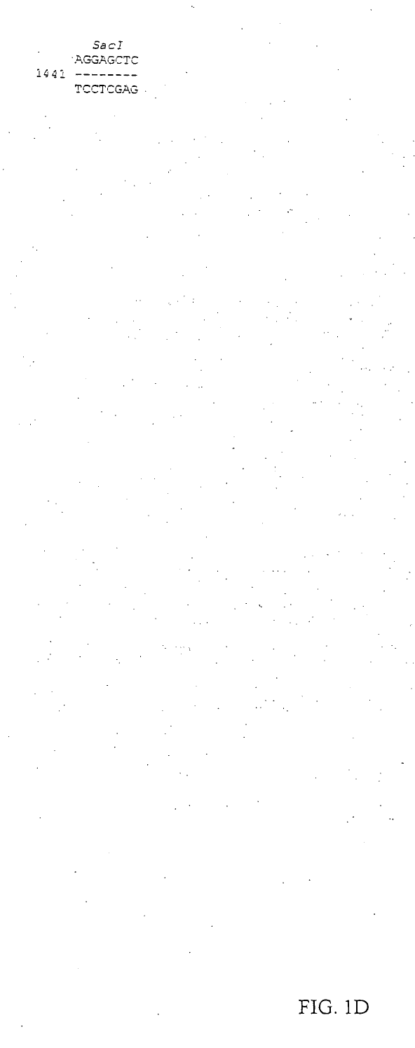

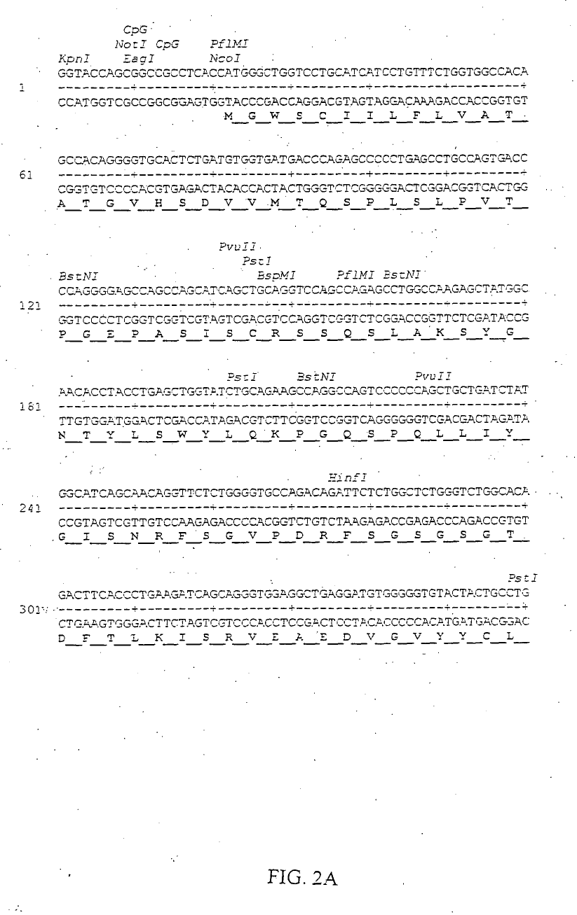

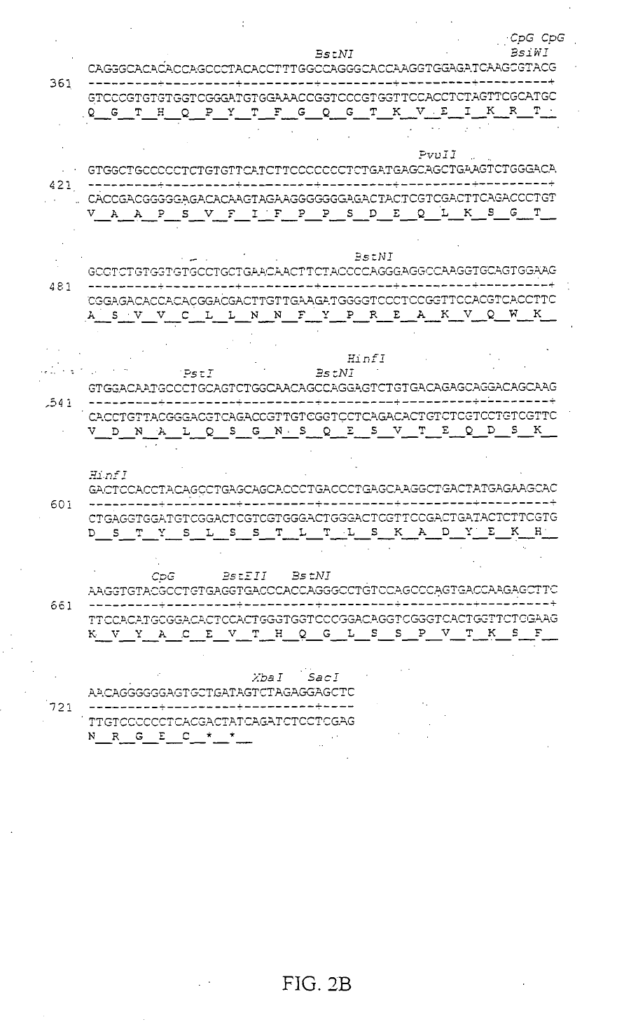

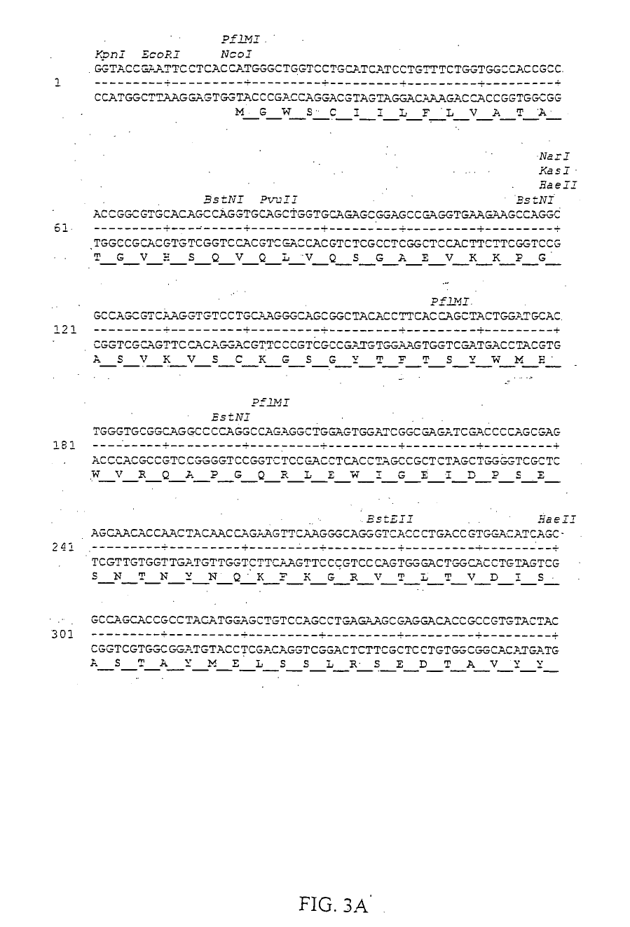

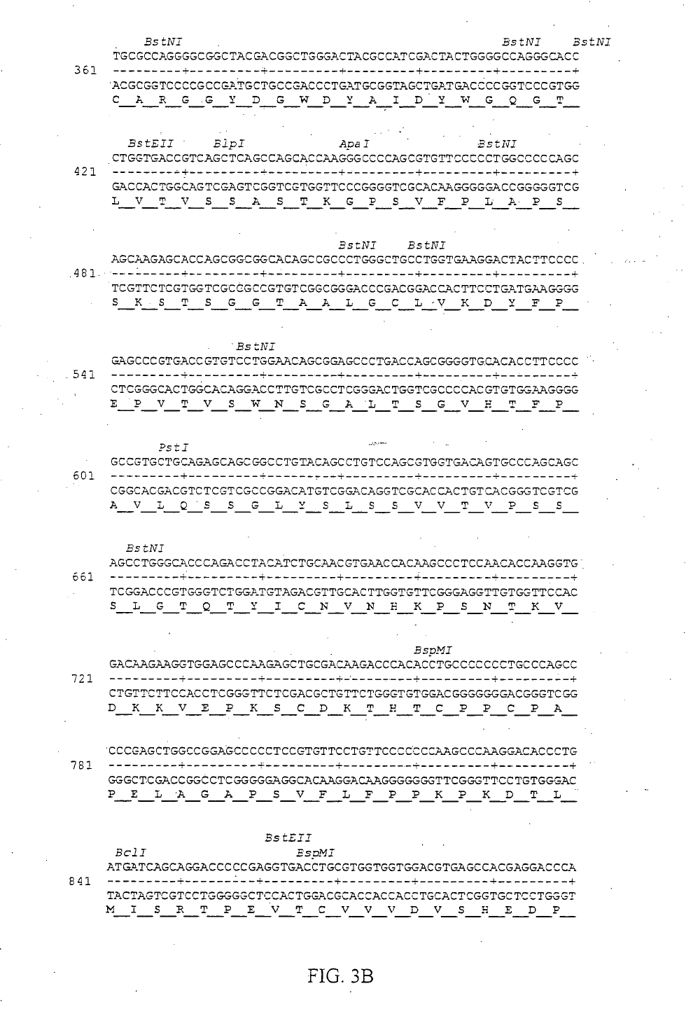

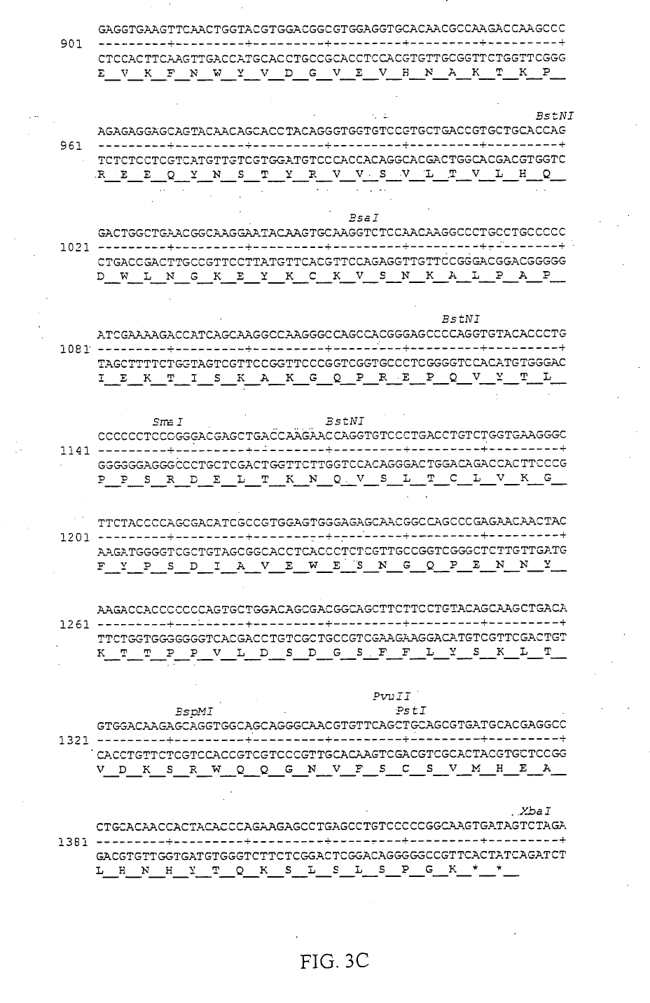

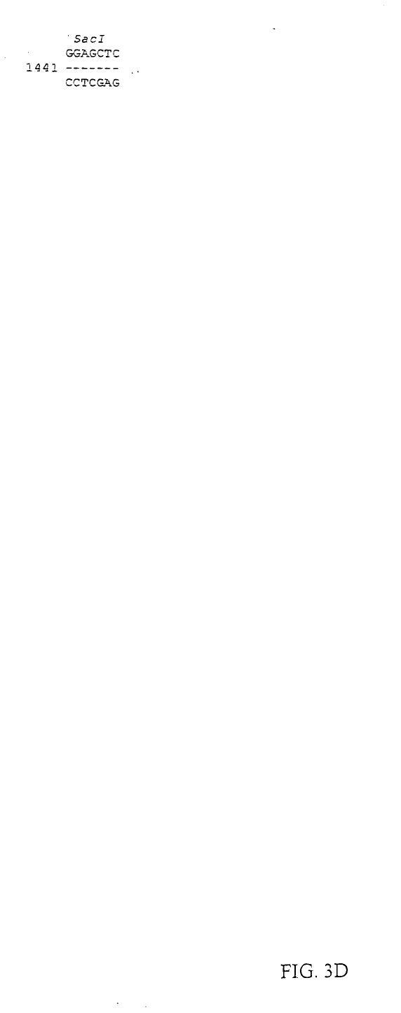

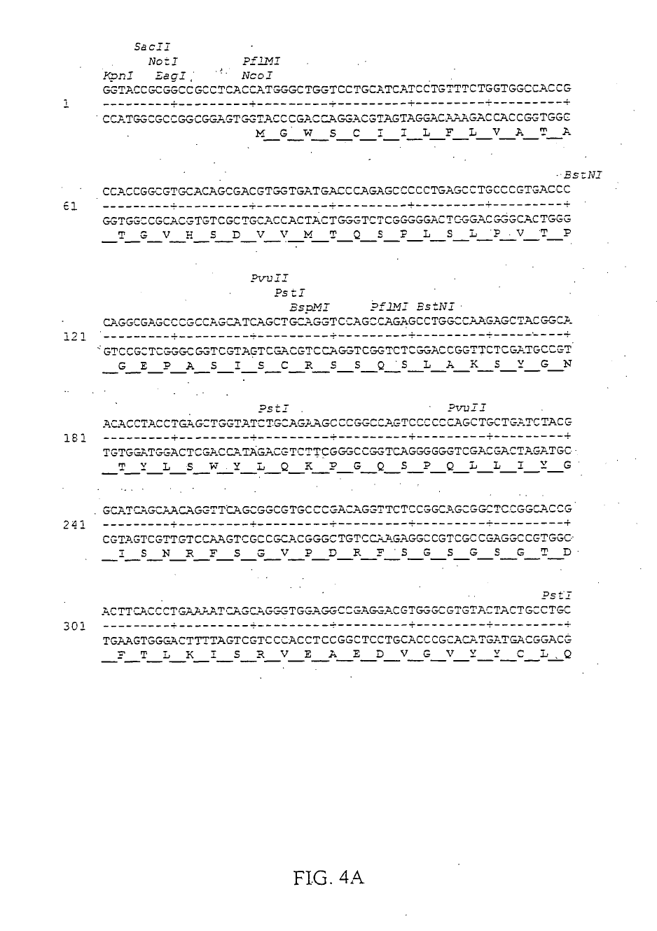

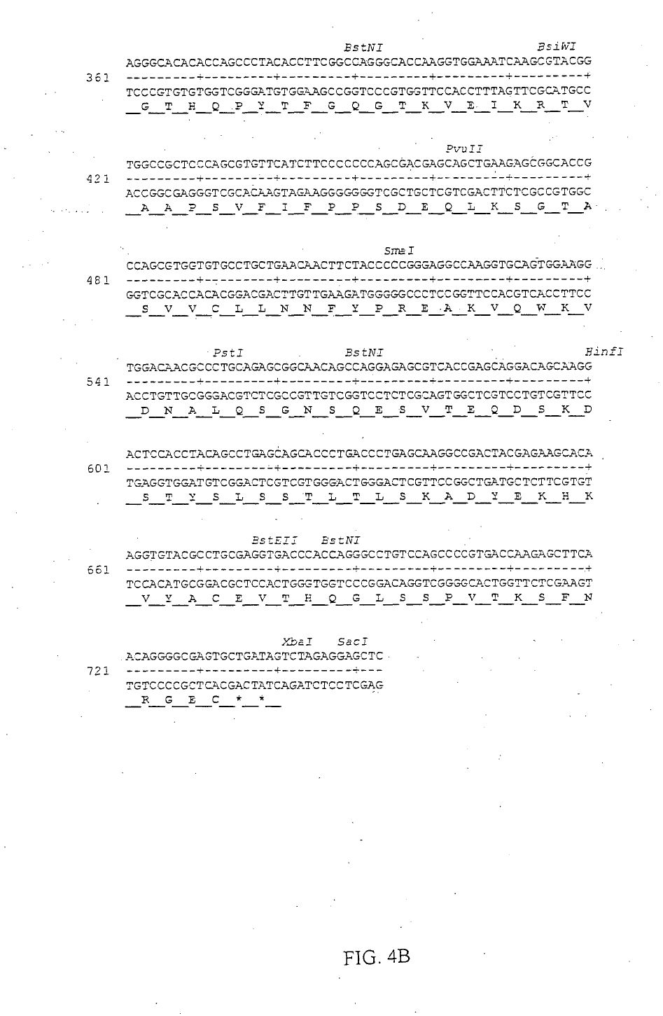

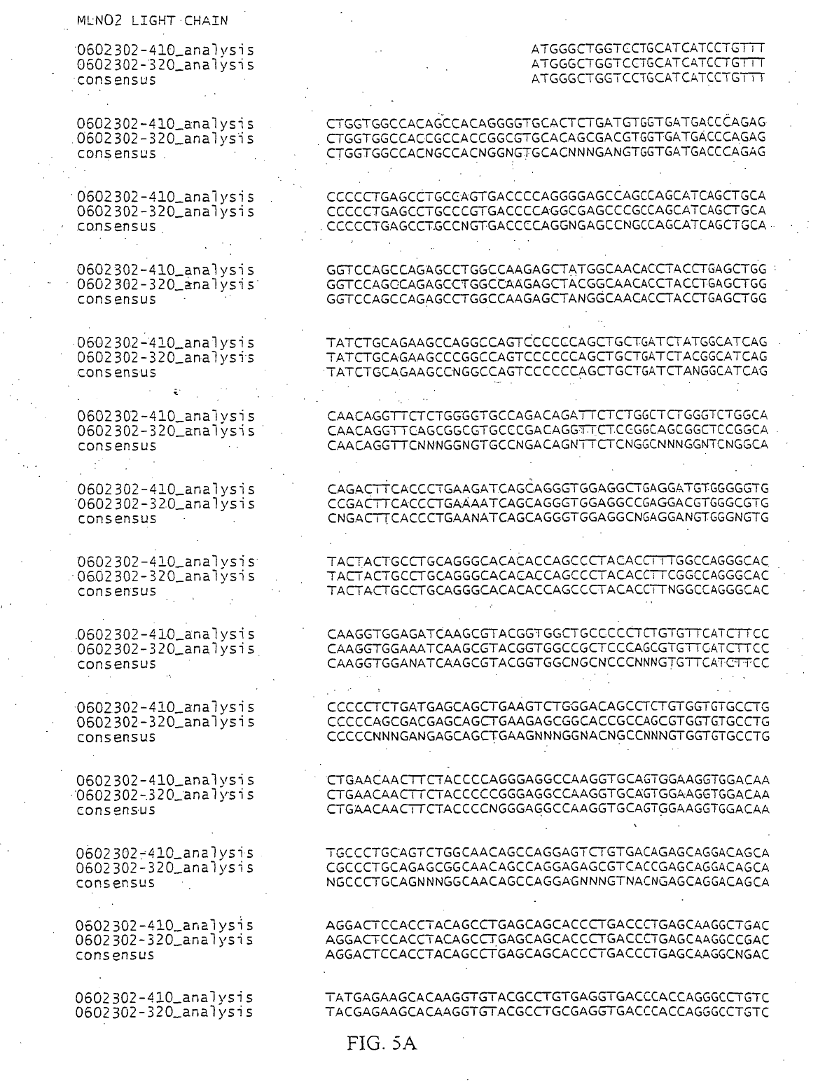

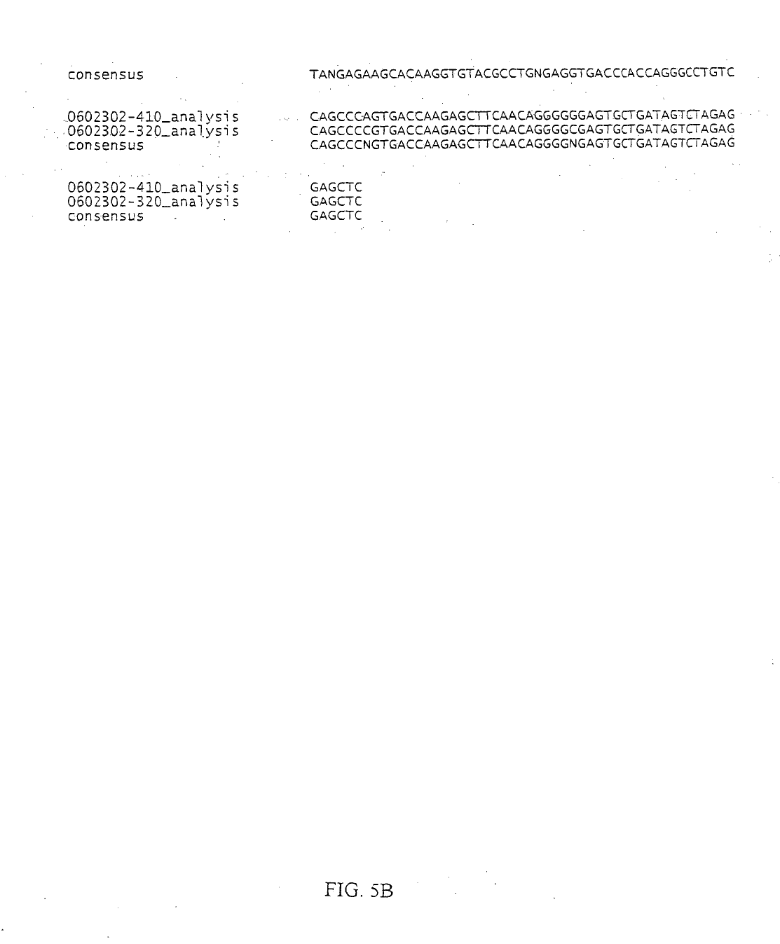

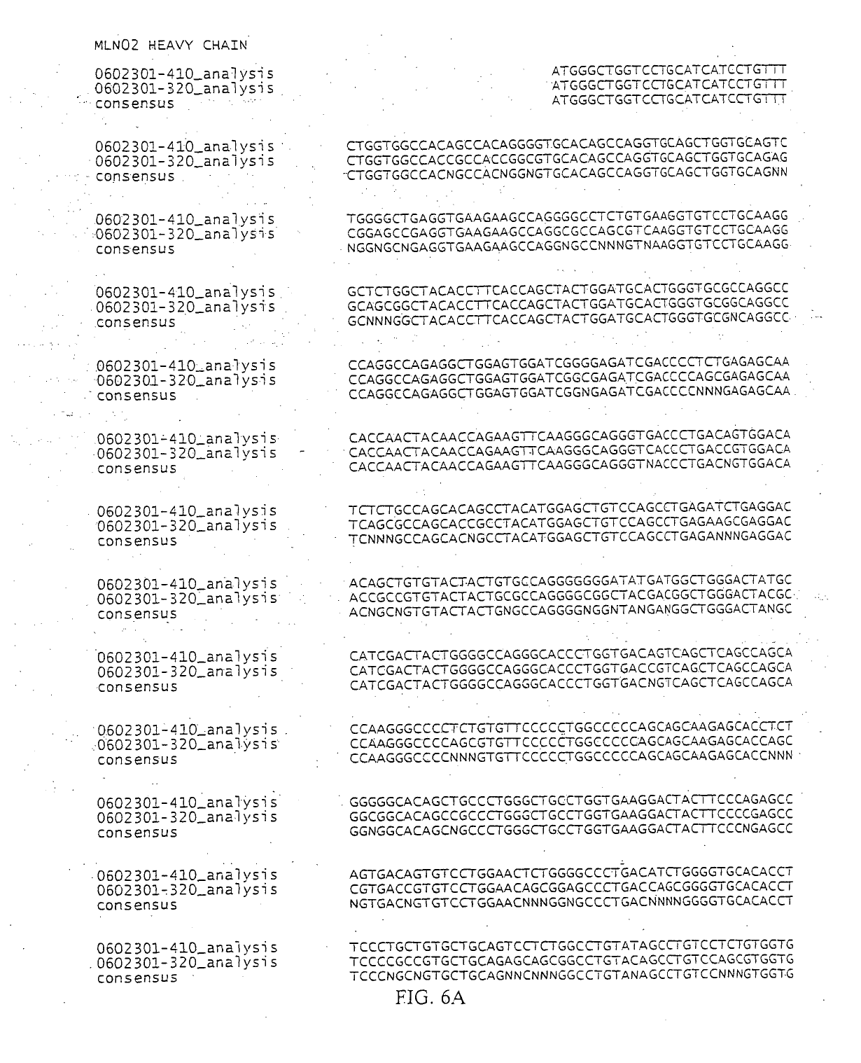

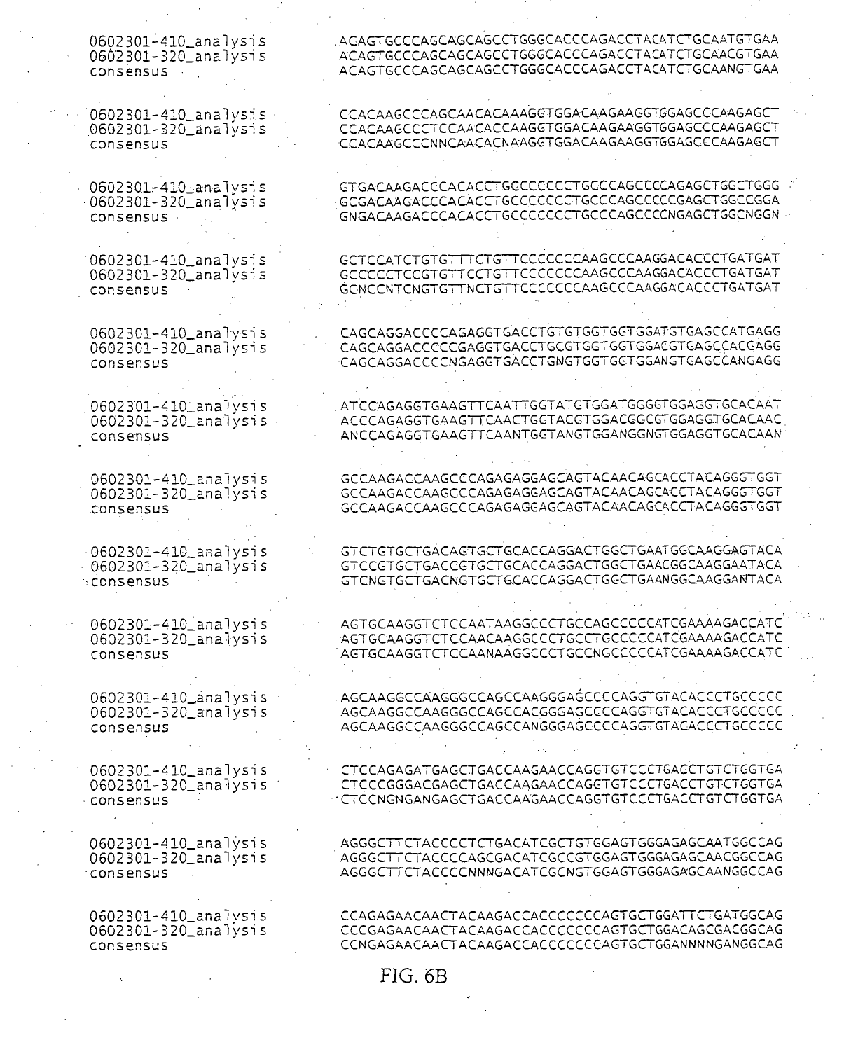

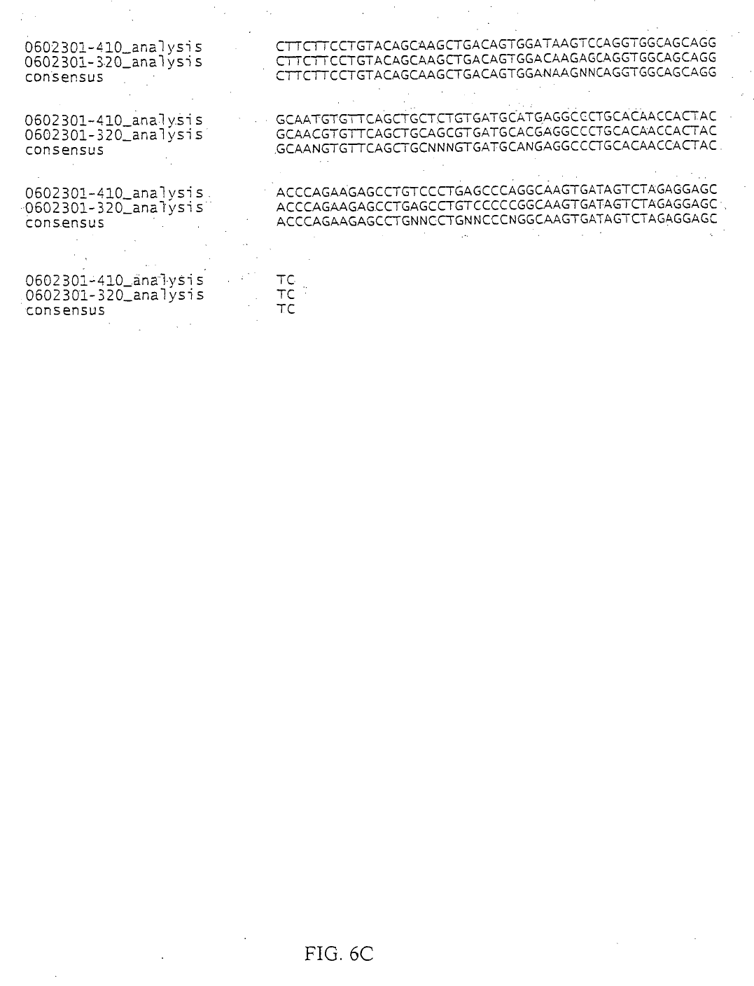

### DETAILED DESCRIPTION OF THE INVENTION

The term “immunoglobulin” as used herein refers to whole antibodies and antigen-binding fragments thereof. Antigen-binding fragments of antibodies include, for example, single chain antibodies, Fv fragments, Fab fragments, Fab′ fragments and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH, domain and hinge region of the heavy chain. Preferred antigen-binding fragments inhibit binding of α4β7 to one or more of its ligands (e.g., the mucosal addressin MAdCAM-1, fibronectin).

The term “humanized immunoglobulin” as used herein refers to an immunoglobulin containing one or more humanized immunoglobulin chains that comprise the heavy chain CDRs (CDR1, CDR2 and CDR3) and light chain CDRs (CDR1, CDR2 and CDR3) of murine Act-1 antibody, and framework and constant regions derived from a light and/or heavy chain of human origin (e.g., CDR-grafted antibodies with or without framework changes). CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al, U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al, U.S. Pat. No. 4,946,778; Huston, U.S. Pat. No. 5,476,786; and Bird, R. E. et al., Science, 242: 423-426 (1998)), regarding single chain antibodies.

Murine ACT-1 Hybridoma cell line, which produces the murine Act-I monoclonal antibody was deposited under the provisions of the Budapest Treaty on Aug. 22, 2001, on behalf of Millennium. Pharmaceuticals, Inc., 75 Sidney Street, Cambridge, Mass. 02139, U.S.A., at the American Type Culture Collection, 10801 University Boulevard, Manassas, Va. 20110-2209, U.S.A., under Accession No. PTA-3663.

MLN02 is a humanized Act-1 immunoglobulin that binds α4β7 integrin (See U.S. application Ser. No. 11/599,151, incorporated herein by reference in its entirety). MLN02 comprises a humanized heavy chain (SEQ ID NO:12) and a humanized light chain (SEQ ID NO:11). The immature humanized heavy chain of MLN02 (amino acids 1-470 of SEQ ID NO:12) comprises a signal peptide (amino acids 1-19 of SEQ ID NO:12), and the mature heavy chain consists of amino acids 20-470 of SEQ ID NO:12. The immature humanized light chain of MLN02 (amino acids 1-238 of SEQ ID NO:11) comprises a signal peptide (amino acids 1-19 of SEQ ID NO:11), and the mature light chain consists of amino acids 20-238 of SEQ ID NO:11.

As described herein, isolated nucleic acids encoding the humanized light chain and the humanized heavy chain of a humanized Act-1 immunoglobulin that has binding specificity for α4β7 integrin have been produced. The isolated nucleic acids of the invention encode the light chain (amino acids 20-238 of SEQ ID NO:11; SEQ ID NO:11) and/or the heavy chain (amino acids 20-470 of SEQ ID NO:12; SEQ ID NO:12) of the humanized antibody MLN02. (See, U.S. patent application Ser. No. 11/599,151). As described herein, the isolated nucleic acids of the invention are different from those disclosed in U.S. Pat. No. 7,147,851 and U.S. application Ser. No. 11/599,151 (each incorporated herein by reference), and comprise nucleotide sequences that provide advantages for expression and production of the encoded antibody or antibody chains.

The isolated nucleic acids of the invention have been designed to contain codon bias for improved expression in Chinese hamster ovary (CHO) cells, to reduce the incidence of sequence elements (e.g., ARE motifs, INS motifs, CRS motifs, cryptic splice donor sites, branch points, internal TATA-boxes, chi-sites, ribosomal entry sites, AT-rich stretches, GC-rich stretches, repeat sequences and RNA secondary structure motifs, certain restriction cites (e.g., BlpI, BsiWI, EcoRI, NotI, PvuK, XbzI)) that can lead to instability, for example, of transfected cell lines or mRNA, and to provide for the inventors desired amount of CpG dinucleotides. The presence of CpG dinucleotides in a construct that encodes protein can prolong the half-life of transcribed mRNA, but CpG dinucleotides also provide sites for methylation of DNA, and methylation can inhibit or suppress transcription of the DNA. Thus, the inclusion of methylation sites in a nucleic acid can lead to instability of host cells that express the nucleic acid, resulting in decreased expression of the nucleic acid (e.g., decreased expression with passage of the cells).

The isolated nucleic acids of the invention, can provide improved expression and production of MLN02 in mammalian cells, such as CHO cells, and be used to produce host cells that stably produce MLN02. Thus, the cost of producing MLN02 can be reduced using the nucleic acids of the invention (e.g., smaller cultures can be used, MLN02 can be produce in higher yield, shorter culture time can be used, and/or the nucleic acids can be used to produce more uniform protein, thereby facilitating downstream processing). Host cells that stably produce MLN02 provide further advantages, such as reducing the possibility that new production methods (e.g., new host cells) will need to be established and obtain regulatory approval.

The isolated nucleic acids of the invention encode the humanized immunoglobulin light chain of the humanized antibody MLN02, the humanized immunoglobulin heavy chain of the humanized antibody MLN02, and/or the humanized immunoglobulin light chain of the humanized antibody MLN02 and the humanized immunoglobulin heavy chain of the humanized antibody MLN02. The isolated nucleic acids of the invention can encode the immature humanized immunoglobulin chains that contain a signal peptide (e.g., SEQ ID NO:1, SEQ ID NO:2, SEQ ID NO:3, SEQ ID NO:4, SEQ ID NO:9; SEQ ID NO:10), or encode the mature humanized immunoglobulin chains that do not contain a signal peptide (e.g., nucleotides 77-1429 of SEQ ID NO:1, 76-1428 of SEQ ID NO:3, 79-735 of SEQ ID NO:2, 78-734 of SEQ ID NO:4, 58-714 of SEQ ID NO:9, 58-1410 of SEQ ID NO:10).

### Nucleic Acids and Recombinant Vectors

The present invention also relates to isolated and/or recombinant (including, e.g., essentially pure) nucleic acids comprising sequences which encode MLN02, the humanized immunoglobulin heavy chain or the humanized immunoglobulin light chain of MLN02.

Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (See e.g., Daugherty, B. L. et al., Nucleic Acids Res., 19(9): 2471 2476 (1991); Lewis, A. P. and J. S. Crowe, Gene, 101: 297-302 (1991)). An isolated nucleic acid can be isolated in a suitable vector, such as a plasmid or viral vector.

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes. “Recombinant” nucleic acids are also those that result from recombination events that occur through the natural mechanisms of cells, but are selected for after the introduction to the cells of nucleic acids designed to allow and make probable a desired recombination event.

The present invention relates more specifically to isolated and/or recombinant nucleic acids comprising a nucleotide sequence which encodes MLN02, the light chain of MLN02 and/or the heavy chain of MLN02.

Nucleic acids of the present invention can be used in the production of MLN02, the light chain of MLN02 and/or the heavy chain of MLN02. For example, a nucleic acid (e.g., DNA) encoding the heavy and/or light chain of MLN02 can be incorporated into a suitable construct (e.g., a recombinant vector) for further manipulation of sequences or for production of the encoded polypeptide in suitable host cells. The nucleic acids can be used to produce the humanized antibody MLN02 in quantities of at least about 0.5 g/L, at least about 1.0 g/L, at least about 1.5 g/L, at least about 1.75 g/L, at least about 2.0 g/L, at least about 2.5 g/L, at least about 2.75 g/L, at least about 3.0 g/L, at least about 4.0 g/L, at least about 4.5 g/L, or at least about 5.0 g/L. For example, in certain embodiments, the nucleic acids of the invention can be expressed in a suitable host cell (e.g., CHO) to produce at least about 0.5 g of MLN02 per liter of culture.

Constructs or vectors (e.g., expression vectors (e.g. pIRES, Clontech)) suitable for the expression of MLN02, or the heavy or light chain of MLN02 are also provided. A variety of vectors are available, including vectors which are maintained in single copy or multiple copy, or which become integrated into the host cell chromosome. The constructs or vectors can be introduced into a suitable host cell, and cells which express MLN02, or the heavy or light chain of MLN02, can be produced and maintained in culture.

Suitable expression vectors, for example mammalian cell expression vectors, can also contain a number of components, including, but not limited to one or more of the following: an origin of replication; a selectable marker gene; one or more expression control elements, such as a transcriptional control element (e.g., a promoter, an enhancer, terminator), and/or one or more translation signals; a signal sequence or leader sequence for membrane targeting or secretion. In a construct or vector, a signal peptide sequence can be provided by the construct or vector or other source. For example, the transcriptional and/or translational signals of an immunoglobulin can be used to direct expression.

A promoter can be provided for expression in a suitable host cell. Promoters can be constitutive or inducible. For example, a promoter can be operably linked to a nucleic acid encoding a humanized immunoglobulin or immunoglobulin chain, such that it directs expression of the encoded polypeptide. A variety of suitable promoters for prokaryotic (e.g., lac, tac, T3, T7 promoters for E. coli) and eukaryotic (e.g., yeast alcohol dehydrogenase (ADH1), SV40, CMV) hosts are available.

In addition, the vectors (e.g., expression vectors) typically comprise a selectable marker for selection of host cells carrying the vector, and, in the case of a replicable vector, an origin of replication. Genes encoding products which confer antibiotic or drug resistance are common selectable markers and may be used in prokaryotic (e.g., β-lactamase gene (ampicillin resistance), Tet gene for tetracycline resistance) and eukaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated.

In one aspect, the invention relates to an isolated nucleic acid encoding the humanized antibody MLN02. The isolated nucleic acid comprises a first nucleotide sequence that encodes the humanized light chain of MLN02 (e.g., amino acids 20-238 of SEQ ID NO:11) and comprises nucleotides 58-714 of SEQ ID NO:9; and a second nucleotide sequence that encodes the heavy chain of MLN02 (e.g. amino acids 20-470 of SEQ ID NO:12) and comprises nucleotides 58-1410 of SEQ ID NO:10. For example, in some embodiments, the first nucleotide sequence comprises nucleotides 77-1429 of SEQ ID NO:1 or nucleotides 76-1428 of SEQ ID NO:3, and/or the second nucleotide sequence comprises nucleotides 79-735 of SEQ ID NO:2 or nucleotides 78-734 of SEQ ID NO:4. In particular embodiments, the first nucleotide sequence comprises nucleotides 58-714 of SEQ ID NO:9 with the proviso that amino acids 20-238 of SEQ ID NO:11 are encoded by said nucleotide sequence; and the second nucleotide sequence comprises nucleotides 58-1410 of SEQ ID NO:10 with the proviso that amino acids 20-470 of SEQ ID NO:12 are encoded by said nucleotide sequence.

In another embodiment, the isolated nucleic acid encodes a mature humanized heavy chain encoded by nucleotides 77-1429 of SEQ ID NO:1 or nucleotides 76-1428 of SEQ ID NO:3; and a mature light chain encoded by nucleotides 79-735 of SEQ ID NO:2 or nucleotides 78-734 of SEQ ID NO:4. If desired, the isolated nucleic acid can encode an immature humanized heavy chain and comprises SEQ ID NO:1 or SEQ ID NO:3, and/or encode an immature humanized light chain and comprises SEQ ID NO:2 or SEQ ID NO:4.

In further embodiments, the invention relates to an isolated nucleic acid that encodes the humanized immunoglobulin light chain of MLN02 (e.g., amino acids 20-238 of SEQ ID NO:11) and comprises nucleotides 58-714 of SEQ ID NO:9. In particular embodiments, the isolated nucleic acid comprises nucleotides 58-714 of SEQ ID NO:9 with the proviso that amino acids 20-238 of SEQ ID NO:11 are encoded by said nucleotide sequence. For example, in particular embodiments, the isolated nucleic acid comprises nucleotides 79-735 of SEQ ID NO:2 or nucleotides 78-734 of SEQ ID NO:4. Preferably, the isolated nucleic acid comprises nucleotides 79-735 of SEQ ID NO:2. If desired, the isolated nucleic acid can further encode a signal sequence. For example, the isolated nucleic acid can comprise SEQ ID NO:2 or SEQ ID NO:4.

In further embodiments, the invention relates to an isolated nucleic acid that encodes the humanized immunoglobulin heavy chain of MLN02 (e.g., amino acids 20-470 of SEQ ID NO:12) and comprises nucleotides 58-1410 of SEQ ID NO:10. In particular embodiments, isolated nucleic acid comprises nucleotides 58-1410 of SEQ ID NO:10 with the proviso that amino acids 20-470 of SEQ ID NO:12 are encoded by said nucleotide sequence. For example, in particular embodiments, the isolated nucleic acid comprises nucleotides 77-1429 of SEQ ID NO:1 or nucleotides 76-1428 of SEQ ID NO:3. Preferably, the isolated nucleic acid comprises nucleotides 77-1429 of SEQ ID NO:1. If desired, the isolated nucleic acid can further encode a signal sequence. For example, the isolated nucleic acid can comprise SEQ ID NO:1 or SEQ ID NO:3.

The invention also relates to recombinant vectors (e.g., expression vectors, such as mammalian cell expression vectors, CHO expression vectors (e.g., pLKTOK38D)) that comprise a nucleic acid encoding the humanized antibody MLN02 (humanized light chain and humanized heavy chain), the humanized heavy chain of MLN02 or the humanized light chain of MLN02. In one embodiment, the recombinant vector encodes (e.g., comprises an isolated nucleic acid encoding) the humanized immunoglobulin light chain of humanized antibody MLN02, and comprises nucleotides 79-735 of SEQ ID NO:2 or nucleotides 78-734 of SEQ ID NO:4. In another embodiment, the recombinant vector encodes (e.g., comprises an isolated nucleic acid encoding) the humanized immunoglobulin heavy chain of the humanized antibody MLN02, and comprises nucleotides 77-1429 of SEQ ID NO:1 or nucleotides 76-1428 of SEQ ID NO:3. If desired, the recombinant vector can further comprise a nucleotide sequence encoding a signal peptide for the light and/or heavy chain. Preferably, the recombinant vector comprises a first nucleotide sequence that encodes the humanized immunoglobulin heavy chain of MLN02 and comprises nucleotides 77-1429 of SEQ ID NO:1, and a second nucleotide sequence that encodes the humanized immunoglobulin light chain of MLN02 and comprises the nucleotide sequence of nucleotides 79-735 of SEQ ID NO:2.

### Method of Producing MLN02

Another aspect of the invention relates to a method of producing the humanized antibody MLN02. The humanized antibody MLN02 can be produced, for example, by the expression of one or more isolated nucleic acids encoding the humanized antibody MLN02 (e.g., encoding the heavy and light chains) in a suitable host cell.

Host cells which produce the humanized antibody MLN02 can be produced using any suitable method. For example, an expression construct (e.g., a mammalian cell expression vector) described herein can be introduced into a suitable host cell, and the resulting cell can be maintained (e.g., in culture, in an animal, in a plant) under conditions suitable for expression of the construct(s) or vector(s). Suitable host cells can be prokaryotic, including bacterial cells such as E. coli (e.g., strain DH5α™ (Invitrogen, Carlsbad, Calif.), B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096), CHO DG44 (Urlaub, G. and Chasin, L A., Proc. Natl. Acad. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The present invention also relates to cells comprising a vector of the invention (e.g., an expression vector): For example, a nucleic acid (i.e., one or more nucleic acids) encoding the heavy and light chains of MLN02, or a construct (i.e., one or more constructs) comprising such nucleic acid(s), can be introduced into a suitable host cell by a method appropriate to the host cell selected (e.g., transformation, transfection, electroporation, infection), such that the nucleic acid(s) are operably linked to one or more expression control elements (e.g., in a vector, in a construct created by processes in the cell, integrated into the host cell genome). Host cells can be maintained under conditions suitable for expression (e.g., in the presence of inducer, suitable media supplemented with appropriate salts, growth factors, antibiotic, nutritional supplements, etc.), whereby the encoded polypeptide(s) are produced. If desired, the encoded protein (e.g., humanized antibody MLN02) can be isolated, for example, from the host cells, culture medium, or milk. This process encompasses expression in a host cell of a transgenic animal or plant (tobacco) (see e.g. WO 92/03918).

If desired, fusion proteins can be produced in which a humanized immunoglobulin or immunoglobulin chain is linked to a non-immunoglobulin moiety (i.e., a moiety which does not occur in immunoglobulins as found in nature) in an N-terminal location, C-terminal location or internal to the fusion protein. For example, some embodiments can be produced by the insertion of a nucleic acid encoding immunoglobulin sequences into a suitable expression vector, such as a pET vector (e.g., pET-15b, Novagen), a phage vector (e.g., pCANTAB 5 E, Pharmacia), or other vector (e.g., pRIT2T Protein A fusion vector, Pharmacia). The resulting construct can be introduced into a suitable host cell for expression. Upon expression, some fusion proteins can be isolated or purified from a cell lysate by means of a suitable affinity matrix (see, e.g., Current Protocols in Molecular Biology (Ausubel, F. M. et al., Eds., Vol. 2, Suppl. 26, pp. 16.4.1-16.7.8 (1991)).

The invention relates to an isolated host cell that comprises an isolated nucleic acid encoding the humanized antibody MLN02 (humanized light chain and humanized heavy chain), the humanized heavy chain of the humanized antibody MLN02 and/or the humanized light chain of the humanized antibody MLN02. For example, in some embodiments, the host cell comprises a recombinant vector (e.g., expression Vector, mammalian expression vector, CHO expression vector) of the invention as referred to herein. In a specific embodiment, the host cell is a CHO cell, such as CHO DG44.

The invention also relates to a method of producing the humanized antibody MLN02, comprising maintaining a host cell of the invention (e.g., a host cell that contains one or more isolated nucleic acids that encode the humanized antibody MLN02 (e.g., a humanized light chain and a humanized heavy chain, a humanized heavy chain, a humanized light chain)) under conditions appropriate for expression of the isolated nucleic acids. For expression of the humanized antibody MLN02, heavy chain, or light chain, a host cell can be maintained under any suitable conditions. For example a host cell can be cultured on a substrate or in suspension. In one embodiment, the host cells are maintained under suitable conditions, such that MLN02 chains are expressed and the humanized antibody MLN02 is produced. In some embodiments, the method further comprises the step of isolating the produced MLN02.

In particular embodiments, the method of producing the humanized antibody

MLN02 results in production of MLN02 in quantities of at least about 0.5 g/L, at least about 1.0 g/L, at least about 1.5 g/L, at least about 1.75 g/L, at least about 2.0 g/L, at least about 2.5 g/L, at least about 2.75 g/L, at least about 3.0 g/L, at least about 4.0 g/L, at least about 4.5 g/L, or at least about 5.0 g/L.

### Example

The CHO DG44 cell line is a double deletion mutant that contains no endogenous copies of the hamster dihydrofolate reductase gene (Som. Cell Molec. Genet. 12:555-666, 1986). A sub-line of these cells that are adapted to grow in suspension in serum-free media, S1-CHO-DG44 cell, was used to make cell lines that produce MLN02. S1-CHO-DG44 cells were thawed and maintained in IS-CHO-V-GS media.

DNA inserts encoding the light and heavy chain of MLN02 (SEQ ID NO:1 and SEQ ID NO:2) were synthesized to include restriction sites for cloning into the expression vector, pTOK59D (see, U.S. Pat. No. 7,053,202). The heavy chain was digested with EcoR I and Xba I restriction sites while the light chain was digested with Not I and Xba I restriction sites for cloning into pTOK59D after the pEFI alpha promoters. The final construct was sequence verified, prepared using Qiagen's EndoFree plasmid DNA Mega kit (Qiagen Cat. No. 12381), and linearized with Pvu I restriction enzyme for transfection.

S1-CHO-DG44 cells (3×10⁶) were transfected at different growth stages (2^{nd} and 3^{rd} day after split) and with different amounts of linearized DNA construct (10 μg, 15 μg, 20 μg, 25 μg, 30 μg and 35 μg). Transfections were performed by electroporation (Bio-Rad Gene Pulser II electroporator, 1000V, 25 υF, and ∞ Ohms). The transfected cells were maintained in IS-CHO-V-GS for 24-48 hours before changing to selection media. 36 transfections were performed to generate 36 transfection pools.

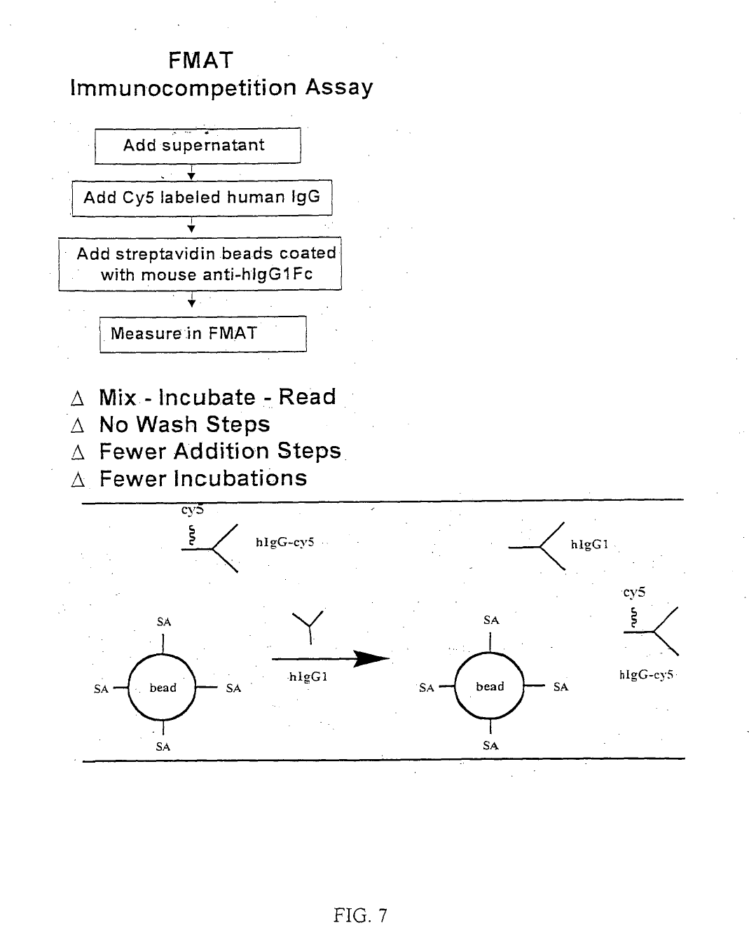

To establish production MLN02 CHO cell line pools, transfected cells were first grown in selection media, αMEM without nucleosides, 10% dialyzed fetal bovine serum, and 0.8 mg/ml G418 (Table 1), for two weeks, and then in double selection media αMEM w/5 nM Methotrexate & 0.8 mg/ml G418 (Table 1) for another 1-2 months. The antibody productivity of each pool was assessed using a human IgG1 hFc Fluid Microvolume Assay Technology (FMAT) immunocompetition assay (FIG. 7, protocol for 96 well plates, Millennium Pharmaceuticals, Inc. Manufacturing Analytical Services). Three stable pools with the highest hFc productivity were identified. These pools were then cloned by limited dilution.

Clones from each pool were isolated by performing limited dilution cloning into 20×96-well tissue culture plates at 0.7 cell/well in double selection medium, αMEM without nucleosides, 10% dialyzed fetal bovine serum, 5 nM methotrexate and 0.8 mg/ml G418 selection media (Table 1). A confluent monolayer of pooled cells was removed from the culture flask using porcine trypsin-EDTA (Invitrogen Cat# 25300-054) and diluted into double selection media for counting and dilution before plating.

The cells were plated in 96-well plates, and were maintained and grown in selection media, αMEM w/5 nM Methotrexate & G418, for 2 weeks without feeding. Then, 50 μl of supernatant from each well was transferred directly into FMAT plates using a Rapidplate 96/384 (Zymark) for the FMAT hFc immunocompetition assay. Single colonies in the 96 well plates with high hFc productivity were identified (about 24-30 clones for each pool), and then expanded sequentially through 24-well cell culture plates and then 6-well cell culture plates. When the clones were at equivalent confluence in the 6-well tissue culture plates, the antibody titer of the clones was measured at 2 different dilutions in the FMAT hFc immunocompetition assay. The 12 clones from each pool with the best antibody titers were chosen and expanded into T-25 flasks in both selection media and Sigma's protein-free, serum-free chemically defined media. The cells in selection media were further expanded into T-75 flasks for freezing.

Based on the antibody titer, 18 production cell lines (clones) were selected for adapting to suspension culture in serum-free Sigma #21 media. A confluent monolayer of cells was removed from the culture flask using porcine trypsin-EDTA (Invitrogen Cat# 25300-054) and subcultured into Sigma #21 media in a new T-25 flasks for suspension growth. Methotrexate was maintained in the medium at 5 nM (Table 1), but G418 was not added due to precipitation, at a cell density of ˜3×10⁵/ml. These cells were then transferred to 125 ml shake flasks to adapt to suspension culture for productivity measurements and freezing cell banks.

The antibody productivity of each selected production cell line was determined by taking cell counts and samples for determining antibody productivity every day for 4 days. The productivities were compared by determining the PCD (Picogram per Cell per Day) of each cell line as well as the final titer at a viability of ˜20%. To measure the PCD, aliquots of the cell culture were collected from the shake flask daily during the log growth phase 2, 3, and 4 days post seeding. The cell density and viability were determined and the hFc concentration in the supernatant was measured using the FMAT immunocompetition assay. The PCD of each cell line was calculated using the formula:

PCD=(Final hFc productivity−Initial hFc productivity)/[log(average cell number)*days].

The PCD's of the high producing cell lines are listed in Table 2. The top producing cell lines were frozen at 5-10×10⁶ cells/ml in 7.5% DMSO, 46.5% fresh Sigma#21 media, 46.5% conditioned Sigma#21 media that the cells were growing in. Aliquots of 1 ml per cryovial were frozen at −80° C. overnight and then transferred to liquid nitrogen for storage.

CHO cell lines for the production of MLN02 have been produced. 5 clones have PCDs over 30 μg/ml/cell/day by FMAT assay. The final titer of clone #27.11 was over 3 mg/ml by FMAT and 1.8 mg/ml by Protein A assay in a shake flask fed-batch culture.

TABLE 1
Media
IS-CHO-V-GS

| | | |
|---|---|---|
| IS-CHO-V-GS | Irvine Scientific, Cat. No. 9198 | 94.90% |
| L-Glutamine | Invitrogen, Cat. No. 25030-081 | 4% |
| Na Pyruvate | Invitrogen, Cat. No. 11360-070 | 1% |
| B-Mercaptoethanol | Invitrogen, Cat. No. 21985-023 | 0.10% |

αMEM w/ 0.8 mg/ml G418

| Dulbecco's Modified | Invitrogen, Cat. No. 32561-037 | 88.90% | |
|---|---|---|---|
| Eagle Medium | | | |
| FBS | Hyclone, SH30079.03IR | 10% | |
| Na Pyruvate | Invitrogen, Cat. No. 11360-070 | 1% | |
| B-Mercaptoethanol | Invitrogen, Cat. No. 21985-023 | 0.10% | |
| Geneticin | Invitrogen, Cat. No. 10131-035 | 0.8 | mg/ml |

αMEM w/ 5 nM Methotrexate & G418

| Dulbecco's Modified | Invitrogen, Cat. No. 32561-037 | 88.90% | |
|---|---|---|---|
| Eagle Medium | | | |
| FBS | Hyclone, SH30079.03IR | 10% | |
| Na Pyruvate | Invitrogen, Cat. No. 11360-070 | 1% | |
| B-Mercaptoethanol | Invitrogen, Cat. No. 21985-023 | 0.10% | |
| Geneticin | Invitrogen, Cat. No. 10131-035 | 0.8 | mg/ml |
| Methotrexate | Calbiochem, Cat. No. 454125 | 5 | mM |

Sigma # 21 w/ 5 nM Methotrexate

| Sigma # 21 | Sigma, Cat. No. C2856-R | 95.90% | |
|---|---|---|---|
| L-Glutamine | Invitrogen, Cat. No. 25030-081 | 3% | |
| Na Pyruvate | Invitrogen, Cat. No. 11360-070 | 1% | |
| B-Mercaptoethanol | Invitrogen, Cat. No. 21985-023 | 0.10% | |
| Methotrexate | Calbiochem, Cat. No. 454125 | 5 | mM |

FBS Freezing Media

| | | |
|---|---|---|
| FBS | Hyclone, SH30079.03IR | 90% |
| DMSO | Sigma, Cat# D2650 | 10% |

TABLE 2
Productivity of MLN02

| | | | | Final |
|---|---|---|---|---|
| | | cell | Final | Titer by |
| | | count at | Titer by | protein A |
| | PCD | day 3 | FMAT | assays |
| Subclone | (ug/ml/cell/day) | (10\hat{ }5/ml) | (ug/ml) | (ug/ml) |
| 21cc.01 | 14.45 | 20.04 | 560 | 550 |
| 21cc.02 | 16.35 | 19.37 | 620 | 640 |
| 21cc.06 | 12.85 | 19.74 | 640 | 640 |
| 21cc.08 | 16.32 | 20.38 | 820 | 820 |
| 21cc.18 | 16.12 | 20.56 | 520 | 530 |
| 21cc.19 | 29.12 | 11.83 | 1040 | 940 |
| 25L.02 | 15.63 | 14.76 | 420 | 420 |
| 25L.03 | 9.44 | 14.1 | 110 | 110 |
| 25L.06 | 10.08 | 16.5 | 140 | 160 |
| 25L.14 | 8.18 | 19.33 | 490 | 480 |
| 27L.01 | 17.5 | 23.67 | 810 | 710 |
| 27L.02 | 29.49 | 21.32 | 800 | 610 |
| 27L.04 | 27.54 | 19.35 | 1190 | 910 |
| 27L.10 | 26.14 | 19.44 | 800 | 690 |
| 27L.11 | 36.45 | 11.95 | 3260 | 1870 |
| 27L.22 | 17.18 | 22.41 | 380 | 380 |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. An isolated nucleic acid encoding the humanized antibody MLN02, comprising a first nucleotide sequence and a second nucleotide sequence, wherein
said first nucleotide sequence encodes the humanized light chain, comprises nucleotides 58-714 of SEQ ID NO:9 and encodes amino acids 20-238 of SEQ ID NO:11; and
said second nucleotide sequence encodes the humanized heavy chain, comprises nucleotides 58-1410 of SEQ ID NO:10 and encodes amino acids 20-470 of SEQ ID NO:12.

2. The isolated nucleic acid of claim 1, wherein said first nucleotide sequence comprises nucleotides 79-735 of SEQ ID NO:2 or nucleotides 78-734 of SEQ ID NO:4.

3. The isolated nucleic acid of claim 1, wherein said second nucleotide sequence comprises nucleotides 77-1429 of SEQ ID NO:1 or nucleotides 76-1428 of SEQ ID NO:3.

4. An isolated nucleic acid encoding the humanized immunoglobulin light chain of the humanized antibody MLN02, wherein said isolated nucleic acid comprises nucleotides 58-714 of SEQ ID NO:9 and encodes amino acids 20-238 of SEQ ID NO:11.

5. The isolated nucleic acid of claim 4, wherein said isolated nucleic acid comprises nucleotides 78-734 of SEQ ID NO:4.

6. The isolated nucleic acid of claim 4, wherein said isolated nucleic acid comprises nucleotides 79-735 of SEQ ID NO:2.

7. An isolated nucleic acid encoding the humanized immunoglobulin heavy chain of the humanized antibody MLN02, wherein said isolated nucleic acid comprises nucleotides 58-1410 of SEQ ID NO:10 and encodes amino acids 20-470 of SEQ ID NO:12.

8. The isolated nucleic acid of claim 7, wherein said isolated nucleic acid comprises nucleotides 76-1428 of SEQ ID NO:3.

9. The isolated nucleic acid of claim 7, wherein said isolated nucleic acid comprises nucleotides 77-1429 of SEQ ID NO:1.

10. A recombinant vector comprising the isolated nucleic acid of claim 1.

11. A recombinant vector comprising the isolated nucleic acid of claim 4.

12. A recombinant vector comprising the isolated nucleic acid of claim 7.

13. The recombinant vector of claim 11, wherein said isolated nucleic acid comprises nucleotides 79-735 of SEQ ID NO:2 or nucleotides 78-734 of SEQ ID NO:4.

14. The recombinant vector of claim 12, wherein said isolated nucleic acid comprises nucleotides 77-1429 of SEQ ID NO:1 or nucleotides 76-1428 of SEQ ID NO:3.

15. A recombinant vector encoding the humanized antibody MLN02, wherein said recombinant vector comprises a first nucleic acid that encodes the heavy chain and a second nucleic acid that encodes the light chain, wherein said first nucleic acid comprises nucleotides 77-1429 of SEQ ID NO:1, and said second nucleic acid comprises nucleotides 79-735 of SEQ ID NO:2.

16. A recombinant vector encoding the humanized antibody MLN02, wherein said recombinant vector comprises a first nucleic acid that encodes the heavy chain and a second nucleic acid that encodes the light chain, wherein said first nucleic acid comprises nucleotides 76-1428 of SEQ ID NO:3, and said second nucleic acid comprises nucleotides 78-734 of SEQ ID NO:4.

17. An isolated host cell comprising the isolated nucleic acid of claim 1.

18. An isolated host cell comprising the recombinant vector of claim 10.

19. An isolated host cell comprising the isolated nucleic acid of claim 4.

20. An isolated host cell comprising the recombinant vector of claim 11.

21. An isolated host cell comprising the isolated nucleic acid of claim 7.

22. An isolated host cell comprising the recombinant vector of claim 12.

23. An isolated host cell comprising the recombinant vector of claim 15.

24. An isolated host cell comprising the recombinant vector of claim 16.

25. A method of producing the humanized antibody MLN02 comprising maintaining a host cell of claim 17 under conditions suitable for expression of a nucleic acid encoding the MLN02 chains, whereby humanized antibody MLN02 chains are expressed and the humanized antibody MLN02 is produced.

26. A method of producing the humanized antibody MLN02 comprising providing the isolated nucleic acid of claim 1 and expressing said isolated nucleic acid, whereby the humanized light and heavy chains are expressed and the humanized antibody MLN02 is produced.

27. A method of producing the humanized antibody MLN02 comprising providing the recombinant vector of claim 15 and expressing said recombinant vector, whereby the humanized light and heavy chains are expressed and the humanized antibody MLN02 is produced.

28. A method of producing the humanized light chain of MLN02 comprising maintaining a host cell of claim 19 under conditions suitable for expression of the nucleic acid encoding the light chain of MLN02, whereby the humanized light chain of MLN02 is expressed and the humanized light chain of MLN02 is produced.

29. A method of producing the humanized light chain of MLN02 comprising providing the isolated nucleic acid of claim 4 and expressing said isolated nucleic acid, whereby the humanized light chain is expressed and the humanized light chain of MLN02 is produced.

30. A method of producing the humanized light chain of MLN02 comprising providing the recombinant vector of claim 11 and expressing said recombinant vector, whereby the humanized light chain is expressed and the humanized light chain of MLN02 is produced.

31. A method of producing the humanized heavy chain of MLN02 comprising maintaining a host cell of claim 21 under conditions suitable for expression of the nucleic acid encoding the heavy chain of MLN02, whereby the humanized heavy chain of MLN02 is expressed and the humanized heavy chain of MLN02 is produced.

32. A method of producing the humanized heavy chain of MLN02 comprising providing the isolated nucleic acid of claim 7 and expressing said isolated nucleic acid, whereby the humanized heavy chain is expressed and the humanized heavy chain of MLN02 is produced.

33. A method of producing the humanized heavy chain of MLN02 comprising providing the recombinant vector of claim 12 and expressing said recombinant vector, whereby the humanized heavy chain is expressed and the humanized heavy chain of MLN02 is produced.
